(19) United States
(12) Patent Application Publication
(10) Pub. No.: US 2015/0306081 A1
(43) Pub. Date: Oct. 29, 2015

(54) Chemotherapy for Drug-Resistant Cancer Cells

(71) Applicants: THE UNIVERSITY OF SYDNEY, New South Wales (AU); ONCOCHEL THERAPEUTICS LLC, Saualito, CA (US)

(72) Inventors: Des Richardson, New South Wales (AU); Patric Jansson, New South Wales (AU); Tetsuo Yamagishi, New South Wales (AU)

(73) Assignee: ONCOCHEL THERAPEUTICS LLC, Sausalito, CA (US)

(21) Appl. No.: 14/646,041

(22) PCT Filed: Nov. 22, 2013
(86) PCT No.: PCT/AU2013/001344

§ 371 Date: May 20, 2015

(30) Foreign Application Priority Data
Nov. 22, 2012 (AU) 2012905123

(51) Int. Cl. A61K 31/444

(52) U.S. Cl. CPC A61K 31/444

(57) ABSTRACT

The present invention relates to new treatments of multidrug resistant disorders, particularly cancers. There is provided a method of treating a cancer including administering an effective amount of a semicarbazone or hydrazone compound to a patient that has cancer, the cancer including a cancerous cell that includes an active efflux mechanism; wherein the compound is a substrate of the active efflux mechanism and the compound is able to form a chelation complex with a metal species in the cancerous cell, the chelation complex being cytotoxic to the cancerous cell.

### FIELD OF THE INVENTION

The invention relates to multidrug resistance and cancer treatment.

### BACKGROUND OF THE INVENTION

Reference to any prior art in the specification is not, and should not be taken as, an acknowledgment or any form of suggestion that this prior art forms part of the common general knowledge in Australia or any other jurisdiction or that this prior art could reasonably be expected to be ascertained, understood and regarded as relevant by a person skilled in the art.

Multidrug resistance (MDR) is one of the major challenges in cancer treatment. One of the best characterized resistance mechanisms in cancer involves cellular efflux of chemotherapeutic drugs through drug pumps, P-glycoprotein (ABCB1), members of the MRP (ABCC) family (MRP1-9) and the ABCG2 (MXR/BCRP) transporter. Considering this, current drug development efforts place an emphasis on chemotherapeutics that are not substrates of such drug efflux pumps to ensure efficient targeting of MDR cells. In addition, in an attempt to increase the accumulation and sensitivity of current chemotherapeutics, various drug resistance modulators have been synthesized to inhibit the function of these efflux pumps. Unfortunately, clinical trials have not yet confirmed the efficacy of these drugs and there is an ever increasing need to develop drugs that effectively target drug resistant cells.

Moreover, it has been demonstrated that MDR can occur through Pgp localized not only on the plasma membrane, but also via lysosomal membrane-bound Pgp through sequestration of Pgp substrate drugs such as doxorubicin (DOX) into the lysosomes. Interestingly, this trapping of DOX within the lysosome protects Pgp expressing cells from DOX accessing its nuclear targets. Hence, these studies indicate another mechanism of MDR that is mediated by lysosomal Pgp in addition to that performed by plasma membrane Pgp.

There remains a need for new approaches to the treatment of MDR in cancer treatment.

### SUMMARY OF THE INVENTION

In one aspect of the invention there is provided a method of treating a cancer including: administering an effective amount of a compound to a patient that has cancer, the cancer including a cancerous cell that includes an active efflux mechanism; wherein the compound is a substrate of the active efflux mechanism and the compound is able to form a chelation complex with a metal species in the cancerous cell, the chelation complex being cytotoxic to the cancerous cell.

There is also provided a method of treating a cancer in an individual including:
administering an effective amount of a compound to an individual who has cancer, the cancer including a cancer cell that includes an active efflux mechanism for efflux of an anti-cancer agent from a cancer cell;
wherein the compound is a substrate of the active efflux mechanism and the compound is able to form a chelation complex with a metal species in the cancer cell, the chelation complex being cytotoxic to the cancer cell.
   thereby treating the cancer in the individual,

In an embodiment, the compound is already in the form of the chelation complex when it enters the cancerous cell.

In an embodiment the active efflux mechanism is mediated by a polypeptide that is a member of the ABC gene superfamily.

In an embodiment the efflux mechanism is mediated by P-glycoprotein (Pgp) (ABCB1). Preferably, the cancer cell expresses Pgp on one or more of the cell surface, endosome and lysosome.

In an embodiment, the method further includes an initial step of; selecting an individual for treatment of cancer, the individual being one who has received chemotherapy or radiotherapy for the cancer. Preferably the cancer is resistant to a chemotherapeutic drug previously administered. More preferably, the cancer is resistant to multiple chemotherapeutic drugs.

In an embodiment the cancer cell has acquired multiple drug resistance, wherein the cancerous cell expresses Pgp on the cell surface, and wherein the compound that is a substrate of the active efflux mechanism used for treatment of the cancer is as described in Formula 1 below.

In an embodiment the individual selected for treatment is selected on the basis of the presence of Pgp on the cell surface of a cancer cell of the individual.

It is preferred that the compound is a compound of Formula 1:

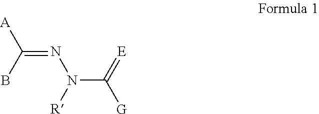

wherein:
A is a monocyclic or polycyclic substituted or unsubstituted 5 or 6-membered heteroaryl group;
B is selected from the group consisting of: a substituted or unsubstituted alkyl group, a substituted or unsubstituted alkene group, a substituted or unsubstituted alkyne group, a monocyclic or polycyclic substituted or unsubstituted cycloalkyl group, a monocyclic or polycyclic substituted or unsubstituted heterocycloalkyl group, a monocyclic or polycyclic substituted or unsubstituted aryl group, or a monocyclic or polycyclic substituted or unsubstituted heteroaryl group;
R¹ is any group that is exchangeable upon binding of the compound to a metal ion (for example, H);

### E is O or S;

G is selected from the group consisting of: a substituted or unsubstituted amine group, a substituted or unsubstituted alkyl group, a substituted or unsubstituted alkene group, a substituted or unsubstituted alkyne group, a monocyclic or polycyclic substituted or unsubstituted cycloalkyl group, a monocyclic or polycyclic substituted or unsubstituted heterocycloalkyl group, a monocyclic or polycyclic substituted or unsubstituted aryl group, or a monocyclic or polycyclic substituted or unsubstituted heteroaryl group.

Preferably A is represented by the Formula 2:

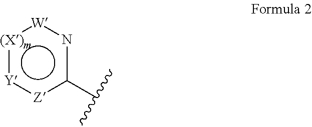

wherein W′, X′, Y′, and Z′ are independently selected from the group consisting of: N, CH, S and O; and wherein the total number of heteroatoms is 1, 2, or 3; and m is 0 or 1. More preferably, A is a substituted or unsubstituted pyridine.

Preferably B is represented by the Formula 3:

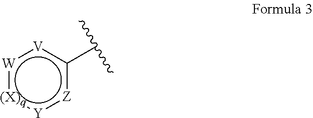

wherein V, W, X, Y, and Z are independently selected from the group consisting of: N, CH, S and O; and wherein the total number of heteroatoms is 1, 2, or 3; and q is 0 or 1. More preferably, B is a substituted or unsubstituted pyridine.

Preferably G is selected from the group consisting of: NH₂, NHR′, or NR′R″, wherein R′ and R″ are independently selected from the group consisting of: a substituted or unsubstituted amine group, a substituted or unsubstituted alkyl group, a monocyclic or polycyclic substituted or unsubstituted heterocycloalkyl group, a monocyclic or polycyclic substituted or unsubstituted aryl group, a monocyclic or polycyclic substituted or unsubstituted cycloalkyl group, a monocyclic or polycyclic substituted or unsubstituted heteroaryl group.

In an embodiment, the compound is a DpT derivative selected from the group consisting of: di-2-pyridylketone 4,4-diphenylcarboxaldehyde semicarbazone (PK44 pH), di-2-pyridylketone 4-methyl-3-thiosemicarbazone (Dp4mT), di-2-pyridylketone 4,4-dimethyl-3-thiosemicarbazone (Dp44mT), di-2-pyridylketone 4-ethyl-3-thiosemicarbazone (Dp4eT), di-2-pyridylketone 4-allyl-3-thiosemicarbazone (Dp4aT), di-2-pyridylketone 4-phenyl-3-thiosemicarbazone (Dp4pT), and di-2-pyridylketone 4,4-diphenylcarboxaldehyde thiosemicarbazone (PK44pTH).

In an alternative embodiment, the compound is a PKIH derivative selected from the group consisting of: di-2-pyridylketone isonicotinoyl hydrazone (PKIH); di-2-pyridylketone benzoyl hydrazone (PKBH), di-2-pyridylketone 4-hydroxybenzoyl hydrazone (PKHH), di-2-pyridylketone 3-bromobenzoyl hydrazone (PBBH), di-2-pyridylketone 4-aminobenzoyl hydrazone (PKAH), di-2-pyridylketone 2-thiophenecarboxaldehyde hydrazone (PKTH), di-2-pyridylketone octanoic hydrazone (PKoctH), di-2 pyridylketone isonicotinoyl thiohydrazone (PKITH), di-2-pyridylketone benzoyl thiohydrazone (PKBTH), di-2-pyridylketone 4-hydroxybenzoyl thiohydrazone (PKHTH), di-2-pyridylketone 3-bromobenzoyl thiohydrazone (PBBTH), di-2-pyridylketone 4-aminobenzoyl thiohydrazone (PKATH), di-2-pyridylketone 2-thiophenecarboxaldehyde thiohydrazone (PKTTH), and di-2-pyridylketone octanoic thiohydrazone (PKoctTH).

Where the compound is intended to form a complex with a copper ion species, preferably the copper ion species is obtained from a salt selected from the group consisting of: CuCl₂, Cu(NO₃)₂, CuSO₄, Cu(OAc)₂, Cu(ClO₄)₂.

Where the compound is intended to form a complex with an iron ion species, preferably the iron ion species is obtained from a salt selected from the group consisting of: FeCl₃, Fe(NO₃)₃, FeSO₄, Fe(OAc)₃, and Fe₂(ClO₄)₃.

In another embodiment there is provided a method for killing a cancer cell that expresses P glycoprotein, including: providing a cancer that that expresses P glycoprotein; contacting the cancer cell with a compound according to Formula 1, or according to Formula 2, or according to Formula 3. In certain embodiments the method includes a step of determining whether the cancer cell expresses P glycoprotein.

In another aspect of the invention there is provided a unit dose treatment product for use in a method of the invention described above.

In another aspect of the invention there is provided a use of an effective amount of a compound in a method of the invention described above.

In another aspect of the invention there is provided a use of an effective amount of a compound in the manufacture of a medicament for use in the method of the invention described above.

In another aspect of the invention there is provided a kit for use in a method of the invention described above, the kit including a compound for administration to a patient. The compound may be provided in the form a unit dose treatment. The compound may be provided in a medicament in an effective amount. The kit may further include a label or package insert with instructions for use.

Further aspects of the present invention and further embodiments of the aspects described in the preceding paragraphs will become apparent from the following description, given by way of example and with reference to the accompanying drawings.

### BRIEF DESCRIPTION OF THE DRAWINGS

FIG. 1. Schematic diagram of mechanism of Pgp-mediated potentiated cytotoxicity by Dp44mT. (1) P-glycoprotein localized on the plasma membrane facilitates transport of some drugs such as Dp44mT into the lysosome to trap and store them to try and prevent the cytoxicity of such agents. (2) As part of endocytosis, P-glycoprotein on the plasma membrane buds inwards to form early endosomes. As a consequence of the process of endocytosis, the topology of P-glycoprotein will be inverted, as shown for other membrane proteins. (3) The inversion of P-glycoprotein leads to the transport of drugs into the vesicle lumen. As the endosome matures into a lysosome, it becomes increasingly acidified. These acidic conditions allow storage and trapping of compounds such as Dp44mT that act like weak bases. (4) In addition, the P-glycoprotein on the lysosomal membrane is functional as it exists under the same conditions as that of plasma membrane P-glycoprotein with catalytic active sites as well as the ATP binding domains still exposed in the cytosol. Therefore, a Pgp substrate such as Dp44mT is not only effluxed by P-glycoprotein on the plasma membrane, but also sequestered into the acidic lysosomes by lysosomal P-glycoprotein pumps. Because of its ionization properties, Dp44mT becomes trapped in acidic lysosomes by becoming positively charged. The Dp44mT then binds copper (stored in lysosomes) to form a redox-active complex that causes permeabilisation of the lysosome and subsequently induces cancer cell death.

FIG. 2. Structures of chemotherapeutic drugs: (A) Dp44mT, (B) Doxorubicin (DOX), (C) Vinblastine (VBL).

FIG. 3. Dp44mT potentiates cytotoxicity in Pgp-expressing cells. (A) Pgp confers resistance to DOX and VBL (IC₅₀/72 h) but can be sensitized in presence of Pgp inhibitors Val (1 μM) and Ela (0.1 μM), only in Pgp expressing KBV1 cells and not in KB31 cells. (B) Dp44mT exerts potentiated cytotoxicity to Pgp expressing KBV1 cells and can be de-sensitized in presence of Pgp inhibitors, Val and Ela while no effect was observed in KB31 cells. (C) Dp44mT exerts potentiated cytotoxicity to Pgp expressing 2008/P200A cells and can be de-sensitized in presence of Pgp inhibitors, Val and Ela (IC₅₀/72 h) while no effect was observed in 2008 cells. (D) Pgp confers resistance to DOX and VBL but can be sensitized in presence of Pgp inhibitors Val and Ela, only in Pgp expressing 2008/P200A cells and not in 2008 cells. (E) Transient knockdown of Pgp using MDR1 siRNA sensitized cytotoxicity to DOX (IC₅₀/72 h) while de-sensitizing cytotoxicity to Dp44mT. Pgp protein expression after knockdown compared to Scr siRNA treated KBV1 cells are shown. Results are mean±SD (3 experiments, at least 4 replicates). ***, versus control, P<0.001

FIG. 4. Dp44mT ligand and its Cu complexes are Pgp substrates. (A) Pgp-mediated ATPase activity induced by compounds relative to basal activity by untreated samples. Verapamil and Val were used as positive controls for substrate and inhibitor of Pgp, respectively. FeCl₃ and CuCl₂ were used as controls for the Fe and Cu complexes with Dp44mT. The fold change was compared to the basal activity where fold change >1 represents ATPase stimulation and <1 represents inhibition. (B) Pgp inhibitors, Val and Ela increased ¹⁴C-Dp44mT (1 μCi) uptake in KBV1 cells but not in KB31 cells (2 h/37° C.). (C) The similar trend was observed for 14C-DOX (1 μCi) and 3H-VBL (1 μCi) uptake. (D)¹⁴C-Dp44mT efflux is higher in Pgp-expressing KBV1 cells relative to KB31 cells. (E) Pgp inhibitors do not affect ¹⁴C-Dp44mT efflux in KB31 cells (F) while in KBV1 cells, repression of ¹⁴C-Dp44mT efflux is observed. Results are mean±SD (3 experiments). ***, versus control, P<0.001

FIG. 5. Accumulation of Rhodamine 123 (Rh123) in KBV1 cells treated with Dp44mT, Ela or Val. (A) Increased accumulation of Rh123 in KBV1 when treated with Dp44mT over various concentrations (30 min/37° C.) while no effect was observed in KB31 cells. (B) Valspodar and (C) Elacridar both increased accumulation of Rh123 at higher concentrations only in Pgp expressing KBV1 cells and not in KB31 cells.

FIG. 6. Lysosomal damage by Dp44mT-Cu and Dp is Pgp dependent. (A) Lysotracker stained (30 min/37° C.) lysosomes are damaged when treated with Cu[Dp44mT] (25 μM). Pgp inhibitors prevented lysosomal damage induced by Cu[Dp44mT] without any changes in cellular morphology. (B) Quantitative analysis using flow cytometry indicated that within 30 min, lysosomal integrity was comprised in Cu[Dp44mT] treated cells which can be reduced in the presence of Pgp inhibitors in Pgp expressing KBV1 cells. Controls including Dp44mT ligand, Val, FeCl₃ and CuCl₂ did not affect the lysosomal staining in the 30 min treatment.

FIG. 7. Val and Ela are not ROS scavengers. Ex vitro DCF assay indicated that neither Val nor Ela are ROS scavengers and that the mechanism by which they save lysosomal damage is not via ROS scavenging abilities of Val or Ela.

FIG. 8. Lysosomal accumulation of Dp44mT is Pgp-dependent. Uptake of ¹⁴C-Dp44mT decreased in the presence of lysosomotropic agents, NH₄Cl (10 mM), MA (100 μM) and CLQ (10 μM) in Pgp-expressing cells KBV1 (A) and 2008/P200A (B) while those that do not express Pgp, KB31 (A) and 2008 cells (B) were not affected.

### DETAILED DESCRIPTION OF THE EMBODIMENTS

The present invention is directed towards a compound that can be used to treat a cancer that includes cancerous cells that have an active efflux mechanism. Active efflux mechanisms assist in the removal of cytotoxic compounds from within a cell. This is particularly problematic when that cytotoxic substance is a compound that is designed to target a cell for therapeutic treatment i.e. an antibiotic or other medicament. This active efflux mechanism is often developed in response to exposure to a compound such as a drug. In this way, an active efflux mechanism is a form of drug resistance. Thus the compound of the present invention can be used to treat a cancer that includes cancerous cells that are drug resistant. It is preferred that the cancerous cells are multidrug resistant (MDR). More preferably, the cancer is an MDR cancer having a drug pump such as P-glycoprotein (Pgp—also known as ABCB1), or any other multidrug resistance drug pump that the compounds are a substrate of (for example, ABCG2, MRPI, etc).

The cancer may be selected from the group including, but not limited to, carcinogenic tumours, turnouts of epithelial origin, such as colorectal cancer, breast cancer, lung cancer, head and neck tumours, hepatic cancer, pancreatic cancer, ovarian cancer, gastric cancer, brain cancer, bladder cancer, prostate cancer and urinary/genital tract cancer; mesenchymal tumours, such as sarcoma; and haemopoietic tumours such as to B cell lymphoma. For example, the cancer may be a haematological tumour, a solid tumour, a non-solid tumour, (e.g., leukaemia, lymphoma).

In brief, the inventors have identified: (1) an additional way of how cancer cells become resistant to cytotoxic drugs by storing them in special vesicles called lysosomes inside tumour cells; and (2) have discovered a way of turning this protective mechanism against the cancer cell to kill it more effectively using the compounds of the invention.

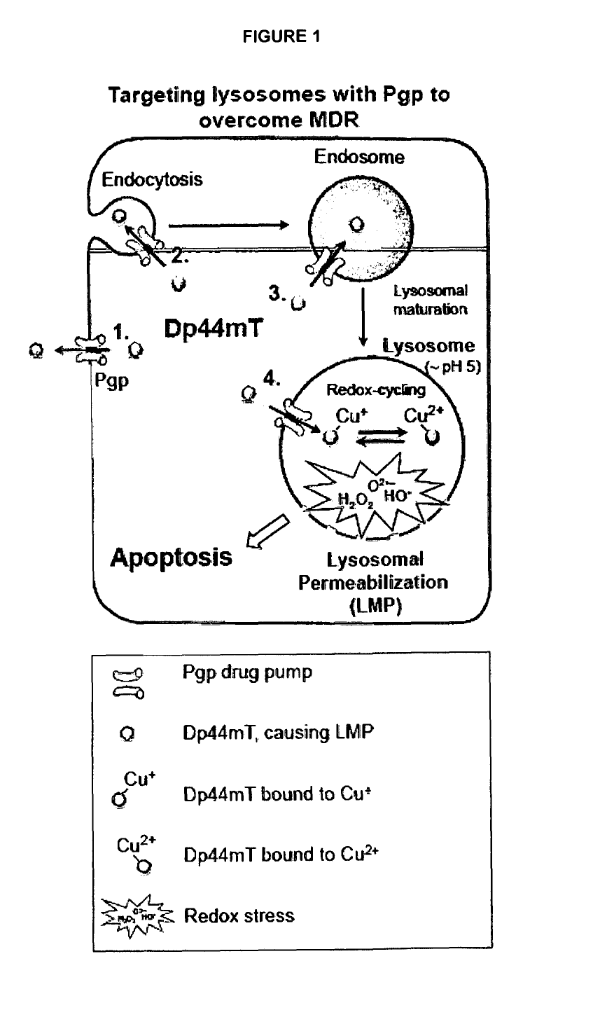

The examples and figures provided illustrate that this process involves intracellular storage of drugs into the special vesicle within the tumour cell known as the “lysosome”. FIG. 1 illustrates the proposed mechanism. The examples show that these lysosomes act to store or trap drugs to protect the cancer cells from damage. We have discovered that this trapping of drugs by the lysosome is mediated by an efflux mechanism, which in this case is a special transport protein, P-glycoprotein (Pgp).

P-glycoprotein acts as a pump and brings the compounds into the storage vesicle (lysosome) of the cancer cell so that they are kept in a safe form, without killing the cancer cells. However, importantly, if these stored compounds generate toxic substances (known as free radicals) inside the lysosomes they can damage the lysosome. This damage causes these storage vesicles to burst, leading to the killing of the tumour cell.

In this way, a mechanism that is supposed to protect the cancer cell against drug-induced damage is being turned against itself to kill the tumour. Therefore, compounds that generate toxic free radicals “hijack” Pgp for their own purpose of killing the cancer. Notably, the examples demonstrate that compounds according to the present invention (e.g. Dp44mT) can exploit this process to selectively target drug-resistant cells.

Without wishing to be bound by theory, the inventors have proposed the following mechanism (see also FIG. 1):
(1) P-glycoprotein localized on the plasma membrane facilitates transport of some drugs such as Dp44mT into the lysosome to trap and store them to try and prevent the cytoxicity of such agents.
(2) As part of endocytosis, Pgp on the plasma membrane buds inwards to form early endosomes. As a consequence of the process of endocytosis, the topology of Pgp will be inverted, as shown for other membrane proteins.
(3) The inversion of Pgp leads to the transport of drugs into the vesicle lumen. As the endosome matures into a lysosome, it becomes increasingly acidified. These acidic conditions allow storage and trapping of compounds such as Dp44mT that act like weak bases.
(4) In addition, the Pgp on the lysosomal membrane is functional as it exists under the same conditions as that of plasma membrane Pgp with catalytic active sites as well as the ATP binding domains still exposed in the cytosol. Therefore, a Pgp substrate (such as Dp44mT) is not only effluxed by Pgp on the plasma membrane, but also sequestered into the acidic lysosomes by lysosomal Pgp pumps. Because of their ionization properties, compounds such as Dp44mT become trapped in acidic lysosomes by becoming positively charged. These charged compounds then bind copper (stored in lysosomes) to form a redox-active metal complex that causes permeabilisation of the lysosome and subsequently induces cancer cell death (see FIG. 1).

While the above is generally discussed in relation to the Pgp system, it will be appreciated that the invention is broadly applicable to other efflux mechanisms. Similarly, while the invention is broadly described in relation to lysosome destruction, the skilled person will appreciate that the cytotoxic compound may target other cell organelles. Similarly, while the preferred embodiments are illustrated in respect of the compound Dp44mT, other compounds exhibiting similar functionality may also be used. These parameters are discussed in more detail in the proceeding sections.

### Compounds of the Invention

The compounds of the present invention are compounds that are substrates of an active efflux mechanism in a cancerous cell. Preferably, the compounds are substrates of drug pumps such as Pgp. It is intended that on entry into a cell, the compounds of the present invention are able to form a chelation-complex with a metal ion species within the cell. The chelation-complex is cytotoxic, and may for example be a reactive oxidation species (ROS) which can cause intracellular damage, potentially leading to cellular death. It is preferred that the chelation-complex is formed within lysosomes, and that the chelation-complex is able to damage the lysosome, causing lysosomal rupture.

Compounds are generally described herein using standard nomenclature. For compounds having asymmetric centres, it will be understood that, unless otherwise specified, all of the optical isomers and mixtures thereof are encompassed. Compounds with two or more asymmetric elements can also be present as mixtures of diastereomers. In addition, compounds with carbon-carbon double bonds may occur in Z and E′forms, with all isomeric forms of the compounds being included in the present invention unless otherwise specified. Where a compound exists in various tautomeric forms, a recited compound is not limited to any one specific tautomer, but rather is intended to encompass all tautomeric forms. Recited compounds are further intended to encompass compounds in which one or more atoms are replaced with an isotope, i.e., an atom having the same atomic number but a different mass number. By way of general example, and without limitation, isotopes of hydrogen include tritium and deuterium and isotopes of carbon include ¹¹C, ¹³C, and ¹⁴C.

Certain compounds are described herein using a general formula that includes variables such as R¹, A, B, G and E. Unless otherwise specified, each variable within such a formula is defined independently of any other variable, and any variable that occurs more than once in a formula is defined independently at each occurrence. Therefore, for example, if a group is shown to be substituted with 0, 1 or 2 R*, the group may be unsubstituted or substituted with up to two R* groups and R* at each occurrence is selected independently from the definition of R*. Also, combinations of substituents and/or variables are permissible only if such combinations result in stable compounds, i.e., compounds that can be isolated, characterized and tested for biological activity.

A “substituent” as used herein, refers to a molecular moiety that is covalently bonded to an atom within a molecule of interest. For example, a “ring substituent” may be a moiety such as a halogen, alkyl group, haloalkyl group or other substituent described herein that is covalently bonded to an atom, preferably a carbon or nitrogen atom, that is a ring member. The term “substituted”, as used herein, means that any one or more hydrogens on the designated atom is replaced with a selection from the indicated substituents, provided that the designated atom's normal valence is not exceeded, and that the substitution results in a stable compound, i.e., a compound that can be isolated, characterized and tested for biological activity. When a substituent is oxo, i.e., ═O, then two hydrogens on the atom are replaced. An oxo group that is a substituent of an aromatic carbon atom results in a conversion of —CH— to —C(═O)— and a loss of aromaticity. For example a pyridyl group substituted by oxo is a pyridone. Examples of suitable substituents are alkyl, heteroalkyl, halogen (for example, fluorine, chlorine, bromine or iodine atoms), OH, ═O, SH, NH₂ (i.e. amine), NH-alkyl (i.e. substituted amine), ═NH, N₃ and NO₂ groups.

The expression “halogen” or “halogen atom” as used herein means fluorine, chlorine, bromine, or iodine.

The term “alkyl” refers to a saturated, straight-chain or branched hydrocarbon group that contains from 1 to 20 carbon atoms, preferably from 1 to 10 carbon atoms, for example a n-octyl group, especially from 1 to 6, i.e. 1, 2, 3, 4, 5, or 6, carbon atoms. Specific examples of alkyl groups are methyl, ethyl, propyl, iso-propyl, n-butyl, iso-butyl, sec-butyl, tert-butyl, n-pentyl, iso-pentyl, n-hexyl and 2,2-dimethylbutyl.

The term “alkenyl” refers to an at least partially unsaturated, straight-chain or branched hydrocarbon group that contains from 2 to 20 carbon atoms, preferably from 2 to 10 carbon atoms, especially from 2 to 6, i.e. 2, 3, 4, 5 or 6, carbon atoms. Specific examples of alkenyl groups are ethenyl (vinyl), propenyl (allyl), iso-propenyl, butenyl, ethinyl, propinyl, butinyl, acetylenyl, propargyl, iso-prenyl and hex-2-enyl group. Preferably, alkenyl groups have one or two double bond(s).

The term “alkynyl” refers to an at least partially unsaturated, straight-chain or branched hydrocarbon group that contains from 2 to 20 carbon atoms, preferably from 2 to 10 carbon atoms, especially from 2 to 6, i.e. 2, 3, 4, 5 or 6, carbon atoms. Specific examples of alkynyl groups are ethynyl, propynyl, butynyl, acetylenyl and propargyl groups. Preferably, alkynyl groups have one or two (especially preferably one) triple bond(s).

The term “cycloalkyl” refers to a saturated or partially unsaturated (for example, a cycloalkenyl group) cyclic group that contains one (or more, in the case of polycyclic) rings (preferably 1 or 2), and contains from 3 to 14 ring carbon atoms, preferably from 3 to 10 (especially 3, 4, 5, 6 or 7) ring carbon atoms. Specific examples of cycloalkyl groups are cyclopropyl, cyclobutyl, cyclopentyl, spiro[4,5]decanyl, norbornyl, cyclohexyl, cyclopentenyl, cyclohexadienyl, decalinyl, bicyclo[4.3.0]nonyl, tetraline, adamantane (i.e. tricycle[3.3.1.1^{3,7}]decane), cyclopentylcyclohexyl and cyclohex-2-enyl.

The term “heterocycloalkyl” refers to a cycloalkyl group as defined above in which one or more (preferably 1, 2 or 3) ring carbon atoms, each independently, have been replaced by an oxygen, nitrogen, silicon, selenium, phosphorus or sulfur atom (preferably by an oxygen, sulfur or nitrogen atom). A heterocycloalkyl group has preferably 1 or 2 rings containing from 3 to 10 (especially 3, 4, 5, 6 or 7) ring atoms (preferably selected from C, O, N and S). Specific examples are piperidyl, prolinyl, imidazolidinyl, piperazinyl, morpholinyl, urotropinyl, pyrrolidinyl, tetra-hydrothiophenyl, tetrahydropyranyl, tetrahydrofuryl and 2-pyrazolinyl group and also lactames, lactones, cyclic imides and cyclic anhydrides.

The term “aryl” refers to an aromatic group that contains one (or more, in the case of polycyclic aryl) rings containing from 6 to 14 ring carbon atoms, preferably from 6 to 10 (especially 6) ring carbon atoms. Examples are phenyl, naphthyl and biphenyl groups.

The term “heteroaryl” refers to an aromatic group that contains one (or more, in the case of polycyclic heteroaryl) rings containing from 5 to 14 ring atoms, preferably from 5 to 10 (especially 5 or 6) ring atoms, and contains one or more (preferably 1, 2, 3 or 4) oxygen, nitrogen, phosphorus or sulfur ring atoms (preferably O, S or N). Examples are pyridyl (for example, 4-pyridyl), imidazolyl (for example, 2-imidazolyl), phenylpyrrolyl (for example, 3-phenylpyrrolyl), thiazolyl, iso-thiazolyl, 1,2,3-triazolyl, 1,2,4-triazolyl, oxadiazolyl, thiadiazolyl, indolyl, indazolyl, tetrazolyl, pyrazinyl, pyrimidinyl, pyridazinyl, oxazolyl, isoxazolyl, triazolyl, tetrazolyl, isoxazolyl, indazolyl, indolyl, benzimidazolyl, benzoxazolyl, benzisoxazolyl, benzthiazolyl, pyridazinyl, quinolinyl, isoquinolinyl, pyrrolyl, purinyl, carbazolyl, acridinyl, pyrimidyl, 2,3′-bifuryl, pyrazolyl (for example, 3-pyrazolyl) and iso-quinolinyl groups.

As used herein a wording defining the limits of a range of length such as, for example, “from 1 to 5” means any integer from 1 to 5, i.e. 1, 2, 3, 4 and 5. In other words, any range defined by two integers explicitly mentioned is meant to comprise and disclose any integer defining said limits and any integer comprised in said range.

It is preferred that compounds for use in the present invention have the general structure shown in Formula 1:

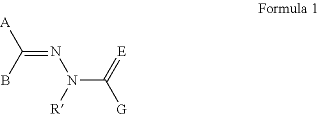

wherein A is a monocyclic or polycyclic substituted or unsubstituted 5 or 6-membered heteroaryl group;

B is selected from the group consisting of: a substituted or unsubstituted alkyl group, a substituted or unsubstituted alkene group, a substituted or unsubstituted alkyne group, a monocyclic or polycyclic substituted or unsubstituted cycloalkyl group, a monocyclic or polycyclic substituted or unsubstituted heterocycloalkyl group, a monocyclic or polycyclic substituted or unsubstituted aryl group, or a monocyclic or polycyclic substituted or unsubstituted heteroaryl group;

R¹ is any group that is exchangeable upon binding of the compound to a metal ion (for example, H);

### E is O, S or NH;

G is selected from the group consisting of: a substituted or unsubstituted amine group, a substituted or unsubstituted alkyl group, a substituted or unsubstituted alkene group, a substituted or unsubstituted alkyne group, a monocyclic or polycyclic substituted or unsubstituted cycloalkyl group, a monocyclic or polycyclic substituted or unsubstituted heterocycloalkyl group, a monocyclic or polycyclic substituted or unsubstituted aryl group, or a monocyclic or polycyclic substituted or unsubstituted heteroaryl group.

Preferred compounds of the invention include semicarbazone and hydrazone compounds. These can be formed, for example, via a condensation reaction of a ketone or aldehyde with a semicarbazide or hydrazide—this is discussed further in the following section. The semicarbazone or hydrazone may be a thiosemicarbazone or thiohydrazone. The terms (thio)semicarbazone and (thio)hydrazone will be used to generally refer to the compounds and indicate that the semicarbazone and hydrazone may or may not contain a thio group (or sulfur atom) as the context requires.

In a preferred form of the invention, the compounds have a di-2-pyridylketone thiosemicarbazone (DpT) type structure represented by Formula 4:

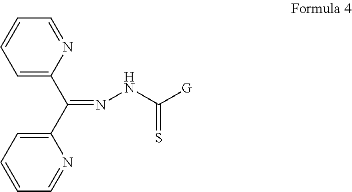

wherein G is as defined herein.

Examples of (thio)semicarbazone derivatives suitable for use in accordance with the present invention include the following: di-2-pyridylketone 4,4-diphenylcarboxaldehyde semicarbazone (PK44 pH), di-2-pyridylketone 4-methyl-3-thiosemicarbazone (Dp4mT), di-2-pyridylketone 4,4-dimethyl-3-thiosemicarbazone (Dp44mT), di-2-pyridylketone 4-ethyl-3-thiosemicarbazone (Dp4eT), di-2-pyridylketone 4-allyl-3-thiosemicarbazone (Dp4aT), di-2-pyridylketone 4-phenyl-3-thiosemicarbazone (Dp4pT), and di-2-pyridylketone 4,4-diphenylcarboxaldehyde thiosemicarbazone (PK44pTH).

Preferred DpT molecules include:

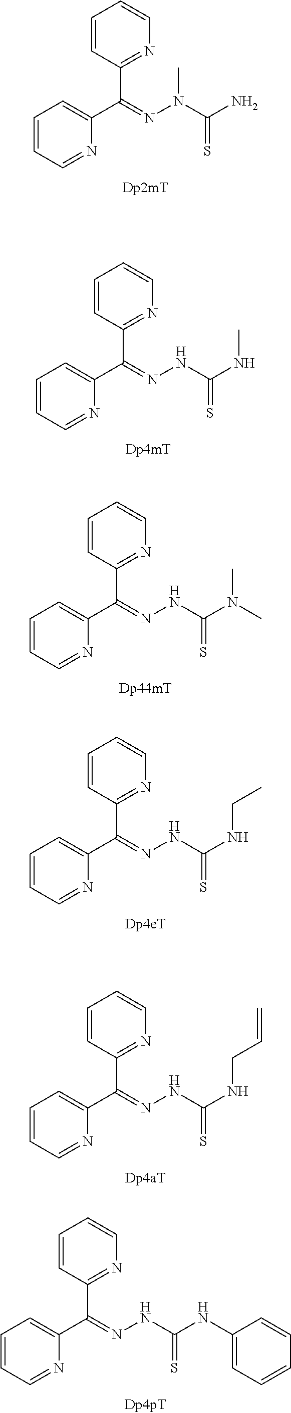 [SMILES: CN(N=C(c1ccccn1)c1ccccn1)C(N)=S ; CNC(=S)NN=C(c1ccccn1)c1ccccn1 ; CN(C)C(=S)NN=C(c1ccccn1)c1ccccn1 ; CCNC(=S)NN=C(c1ccccn1)c1ccccn1 ; C=CCNC(=S)NN=C(c1ccccn1)c1ccccn1 ; S=C(NN=C(c1ccccn1)c1ccccn1)Nc1ccccc1]

It will be appreciated that molecules exhibiting similar structures or minor structural variations may also be appropriate. In some embodiments, the thio moiety may be replaced with an oxygen atom or NH group.

In an alternative preferred form of the invention the compounds have a di-2-pyridylketone isonicotinoyl hydrazone (PKIH) type structure represented by Formula 5:

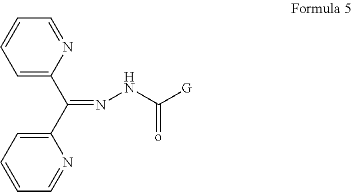

wherein G is as defined herein.

Examples of (thio)hydrazone derivatives suitable for use in accordance with the present invention include the following: di-2-pyridylketone isonicotinoyl hydrazone (PKIH); di-2-pyridylketone benzoyl hydrazone (PKBH), di-2-pyridylketone 4-hydroxybenzoyl hydrazone (PKHH), di-2-pyridylketone 3-bromobenzoyl hydrazone (PBBH), di-2-pyridylketone 4-aminobenzoyl hydrazone (PKAH), di-2-pyridylketone 2-thiophenecarboxaldehyde hydrazone (PKTH), di-2-pyridylketone octanoic hydrazone (PKoctH), di-2 pyridylketone isonicotinoyl thiohydrazone (PKITH), di-2-pyridylketone benzoyl thiohydrazone (PKBTH), di-2-pyridylketone 4-hydroxybenzoyl thiohydrazone (PKHTH), di-2-pyridylketone 3-bromobenzoyl thiohydrazone (PBBTH), di-2-pyridylketone 4-aminobenzoyl thiohydrazone (PKATH), di-2-pyridylketone 2-thiophenecarboxaldehyde thiohydrazone (PKTTH), and di-2-pyridylketone octanoic thiohydrazone (PKoctTH).

In a preferred embodiment, G is selected from:

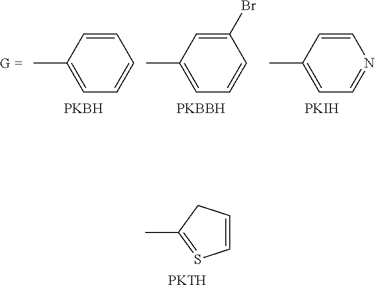

It will be appreciated that molecules exhibiting similar structures or minor structural variations may also be appropriate. In some embodiments, the oxygen atom may be replaced with a sulphur atom or NH.

Metal ion complexes (i.e. “chelation complexes with a metal species”) in accordance with the present invention may be formed when compounds come into contact with a metal ion species. The metal ion complexes may also be preformed metal ion complexes i.e. complexes formed before exposure of the cancer cell/tumour to the compounds. Suitable metal ion salts include, but are not limited to, metal halides, nitrates, sulfates, perchlorates, acetates, and triflates. Suitable metal ion species include copper and iron species and any metal ions that are able to form a complex with the compounds discussed herein. A particularly suitable class is transition metal ions. As used herein, the term “transition metal ion” refers to an element whose atom has an incomplete d sub-shell, or which can give rise to cations with an incomplete d sub-shell. Metal ions that may be used are selected from the group consisting of rhodium, scandium, titanium, vanadium, chromium, ruthenium, platinum, manganese, iron, cobalt, nickel, copper, molybdenum and zinc ions. Particularly preferred metal ions are iron and copper ions. The iron may have an oxidation state of II or III, and the copper may have an oxidation state of II.

(Thio)semicarbazone and (thio)hydrazone compounds of the invention in accordance with the present invention may function as tridentate ligands capable of forming metal ion complexes.

Iron complexes in accordance with the present invention include Fe[PKIH]₂, Fe[PKBH]₂, Fe[PKBBH]₂, Fe[PKHH]₂, Fe[PBBH]₂, Fe[PKAH]₂, Fe[PKTH]₂, Fe[PK44 pH]₂, Fe[PKoctH]₂, Fe[DpT]₂, Fe[Dp4mT]₂, Fe[Dp44mT]₂, Fe[Dp4eT]₂, Fe[Dp4aT]₂, and Fe[Dp4pT]₂. The iron ion may be Fe(II) or Fe(III). Suitable iron salts for forming iron complexes include, but are not limited to, FeCl₃, Fe(NO₃)₃, FeSO₄, Fe(OAc)₃, and Fe₂(ClO₄)₃.

Copper complexes in accordance with the present invention include Cu[PKIH]₂, Cu[PKBH]₂, Cu[PKBBH]₂, Cu[PKHH]₂, Cu[PBBH]₂, Cu[PKAH]₂, Cu[PKTH]₂, Cu[PK44 pH]₂, Cu[PKoctH]₂, Cu[DpT]₂, Cu[Dp4mT]₂, Cu[Dp44mT]₂, Cu[Dp4eT]₂, Cu[Dp4aT]₂, and Cu[Dp4pT]₂. Suitable copper salts for forming copper complexes include, but are not limited to, CuCl₂, Cu(NO₃)₂, CuSO₄, Cu(OAc)₂ and Cu(ClO₄)₂.

General examples of an iron complex and a copper complex are provided below:

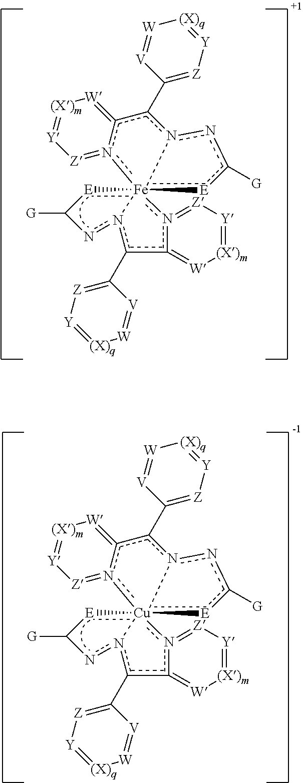

As a more specific example, an iron complex and a copper complex of the ligand PKAH is illustrated below:

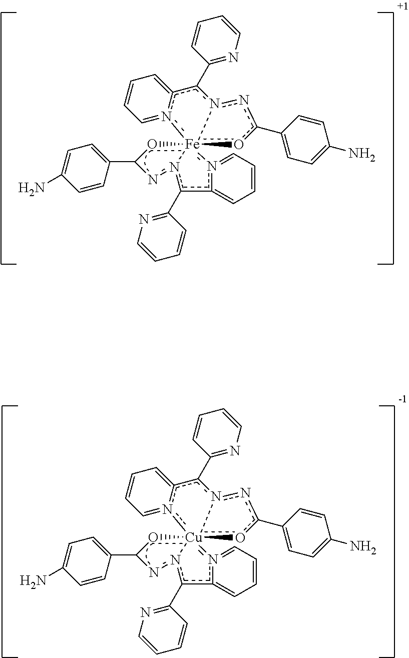

In another example, an iron complex and a copper complex of the ligand Dp4mT is illustrated below:

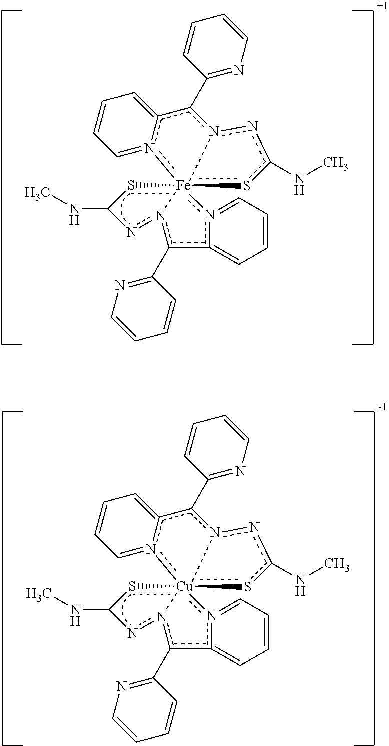

The metal ion complexes of DpT or PKIH ligands may be neutral or charged.

### Synthesis of Compounds

Suitable methods for the synthesis of compounds of the invention are well known to those skilled in the art and some are described, for example, in J. March, Advanced Organic Chemistry, 4th Edition (John Wiley & Sons, New York, 1992); and Vogel's Textbook of Practical Organic Chemistry, 5th Edition (John Wiley & Sons, New York, 1989).

Compounds of the invention may be prepared by means of a Schiff base condensation reaction in which a ketone or aldehyde is condensed with either an acid (thio)hydrazide or acid (thio)semicarbazide to produce a corresponding (thio)semicarbazone or (thio)hydrazone compound.

Suitable synthetic methods of producing (thio)semicarbazone and (thio)hydrazone compounds have been described for example by Bacchi^{1,2} and Johnson.³

An example of a general synthetic route for preparing analogues of the invention, having a di-2-[heterocycle]ketone thiosemicarbazone structure is represented in the following general reaction scheme:

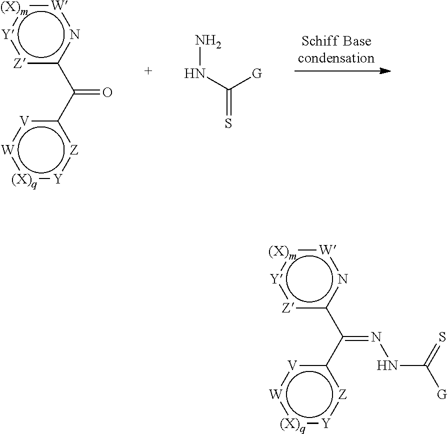

An example of a synthetic route for preparing the compound Dp4eT is illustrated below:

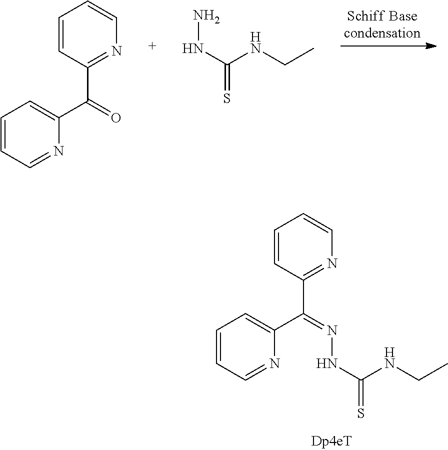

A typical example of a general synthetic route for preparing analogues of the invention having a di-2-[heterocycle]ketone isonicotinoyl hydrazone structure is represented in the following reaction scheme:

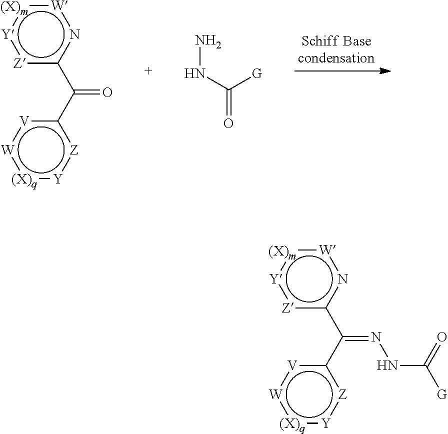

An example of a synthetic route for preparing the compound PK44 pH is illustrated below:

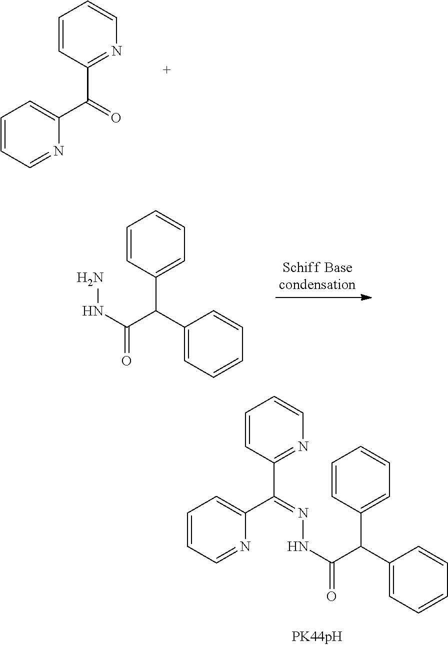 [SMILES: O=C(c1ccccn1)c1ccccn1 ; NNC(=O)C(c1ccccc1)c1ccccc1 ; O=C(NN=C(c1ccccn1)c1ccccn1)C(c1ccccc1)c1ccccc1]

The condensation reactions represented above may be carried out under conditions known to those skilled in the art. For example, suitable solvent systems include ethanol, methanol, ethanol/water, methanol/water, or other common organic solvents such as acetone, benzene, toluene, etc. Compounds of the present invention may be purified using standard techniques, including recrystallisation from a suitable solvent, and column chromatography.

In an alternative synthesis, thiosemicarbazones and thiohydrazones may be prepared from the corresponding semicarbazone and hydrazone compounds respectively, using methods known to those skilled in the art. For example, a C═O group may be converted into a C═S group using Lawesson's reagent under standard conditions. For example, a hydrazone compound of the present invention may be heated at reflux with Lawesson's reagent in a suitable solvent, such as toluene or benzene, to produce the corresponding thio-compound. Preparation of compounds of the present invention is illustrated in the following general scheme:

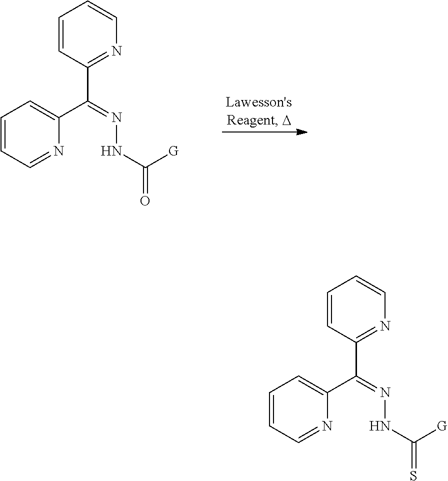

Suitable methods for forming chelation complexes can be found in Richardson¹⁸, Kalinowski⁹, Bernhardt¹⁹ and Jansson.²⁰

### Preparation of Medicaments, Treatment Regimes and Dosages

In accordance with the present invention, when used for the treatment of a drug resistant cancer, the compound(s) of the invention may be administered alone or in combination with other agents as part of a therapeutic regimen. The compounds may be administered as a pharmaceutical or veterinary formulation which comprises at least one compound according to the invention.

Pharmaceutical compositions suitable for the delivery of compounds of the present invention and methods for their preparation will be readily apparent to those skilled in the art. Such compositions and methods for their preparation may be found, for example, in Remington's Pharmaceutical Sciences. 19th Edition (Mack Publishing Company, 1995).

In other embodiments the compound(s) of the present invention may be formulated in combination with one or more other therapeutic agents.

In other embodiments of the present invention, the compounds of the invention may be included in combination treatment regimens with surgery and/or other known treatments or therapeutic agents, such as other anticancer agents, in particular, chemotherapeutic agents, radiotherapeutic agents, and/or adjuvant or prophylactic agents. Suitable agents are listed, for example, in the Merck Index, An Encyclopaedia of Chemicals, Drugs and Biologicals, 12th Ed., 1996, the entire contents of which are incorporated herein by reference.

For example, when used in the treatment of solid tumours, compounds of the present invention may be administered with one or more chemotherapeutic agents or combinations thereof, such as: adriamycin, taxol, docetaxel, fluorouracil, melphalan, cisplatin, alpha interferon, COMP (cyclophosphamide, vincristine, methotrexate and prednisone), etoposide, mBACOD (methotrexate, bleomycin, doxorubicin, cyclophosphamide, vincristine and dexamethasone), PROMACE/MOPP (prednisone, methotrexate (w/leucovin rescue), doxorubicin, cyclophosphamide, taxol, etoposide/mechlorethamine, vincristine, prednisone and procarbazine), vincristine, vinblastine, angioinhibins, TNP-470, pentosan polysulfate, platelet factor 4, angiostatin, LM-609, SU-101, CM-101, Techgalan, thalidomide, SP-PG, and the like. Other examples of anticancer agents include alkylating agents such as nitrogen mustards (e.g. mechlorethamine, melphalan, chlorambucil, cyclophosphamide, (L-sarcolysin), and ifosfamide), ethylenimines and methylmelamines (e.g. hexamethylmelamine, thiotepa), alkylsulfonates (e.g. busulfan), nitrosoureas (e.g. carmustine, lomustine, semustine, streptozocin), triazenes (e.g. dacarbazine (dimethyltriazeno-imidazolecarboxamide), temozolomide), folic acid analogues (e.g. methotrexate), pyrimidine analogues (e.g. 5-fluorouricil, floxuridine, cytarabine, gemcitabine), purine analogues (e.g. 6-mercaptopurine, 6-thioguanine, pentostatin, (2′-deoxycoformycin) cladribine, fludarabine, vinca alkaloids (e.g. vinblastine, vincristine), taxanes (e.g. paclitaxel, docetaxel), epipodophyllotoxins (e.g. etoposide, teniposide), camptothecins (e.g. topotecan, irinotecan), antiobiotics (e.g. actinomycin D, daunorubicin (e.g. daunomycin, rubidomycin), doxorubicin, bleomycin, mitomycin C, methramycin), enzymes (e.g. L-asparaginase), interferon-alpha, interleukin-2, cisplatin, carboplatin, mitoxantrone, hydroxyurea, procarbazine, mitotane, aminoglutethimide, imatinib, adrenocorticosteroids (e.g. prednisone), progestins (e.g. hydroxyprogesterone caproate, medroxyprogesterone acetate, megestrol acetate), estrogens (e.g. diethylstilbestrol, ethinyl estradiol), antiestrogens (e.g. tamoxifen, anastrozole), androgens (e.g. testosterone propionate, fluoxymesterone), antiandrogens (e.g. flutamide), and gonadotropin-releasing hormone analogues (e.g. leuprolide).

In particularly preferred embodiments one more compounds of the invention may be used in combination with gemcitabine or 5-fluorouracil, or in combination with gemcitabine and 5-fluorouracil.

Combination regimens may involve the active agents being administered together, sequentially, or spaced apart as appropriate in each case. Combinations of active agents including compounds of the invention may be synergistic.

The compound(s) may also be present as suitable salts, including pharmaceutically acceptable salts. By pharmaceutically acceptable salt it is meant those salts which, within the scope of sound medical judgement, are suitable for use in contact with the tissues of humans and lower animals without undue toxicity, irritation, allergic response and the like, and are commensurate with a reasonable benefit/risk ratio. Pharmaceutically acceptable salts are well known in the art. Acid addition salts, such as hydrochloride salts, are a particularly preferred embodiment of the invention.

For example, suitable pharmaceutically acceptable salts of compounds according to the present invention may be prepared by mixing the compounds of the invention with a pharmaceutically acceptable acid (including inorganic and organic acids) or a pharmaceutically acceptable base (including inorganic and organic bases). Suitable pharmaceutically acceptable salts of the compounds of the present invention therefore include acid addition salts and base salts. For example, Berge⁴ describes pharmaceutically acceptable salts in detail and a review on suitable salts is provided by Handbook of Pharmaceutical Salts: Properties, Selection, and Use by Stahl and Wermuth (Wiley-VCH, Weinheim, Germany, 2002).

Suitable pharmaceutically acceptable acids include but are not limited to acetic acid, benzenesulfonic acid, benzoic acid, camphorsulfonic acid, citric acid, ethenesulfonic acid, fumaric acid, gluconic acid, glutamic acid, hydrobromic acid, hydrochloric acid, isethionic acid, lactic acid, maleic acid, malic acid, malonic acid, mandelic acid, methanesulfonic acid, mucic acid, nitric acid, pamoic acid, pantothenic acid, phosphoric acid, oxalic acid, succinic acid, sulfuric acid, tartaric acid acid, p-toluenesulfonic acid, and the like. Preferred acid addition salts are hydrochloric, hydrobromic, phosphoric, and sulfuric salts, and most particularly preferred is the hydrochloric salt.

Suitable pharmaceutically acceptable base salts include aluminium, arginine, benzathine, calcium, choline, diethylamine, diolamine, glycine, lysine, magnesium, meglumine, olamine, potassium, sodium, tromethamine and zinc salts.

Suitable pharmaceutically acceptable salts of PKIH and DpT analogues may be prepared by mixing the compounds of the invention with a pharmaceutically acceptable acid (including inorganic and organic acids) or a pharmaceutically acceptable base (including inorganic and organic bases). Suitable pharmaceutically acceptable salts of the compounds of the present invention therefore include acid addition salts and base salts.

Hemisalts of acids and bases may also be formed, for example, hemisulphate and hemicalcium salts.

The salts can be prepared in situ during the final isolation and purification of the compounds of the invention, or separately by reacting the free base function with a suitable organic acid. Representative acid addition salts include acetate, adipate, alginate, ascorbate, asparate, benzenesulfonate, benzoate, bisulfate, borate, butyrate, camphorate, camphorsulfonate, citrate, cyclopentanepropionate, digluconate, dodecylsulfate, ethanesulfonate, fumarate, glucoheptonate, glycerophosphate, hemisulfate, heptonate, hexanoate, hydrobromide, hydrochloride, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, toluenesulfonate, undecanoate, valerate salts, and the like. Representative alkali or alkaline earth metal salts include sodium, lithium, potassium, calcium, magnesium, and the like, as well as nontoxic ammonium, quaternary ammonium, and amine cations, including, but not limited to ammonium, tetramethylammonium, tetraethylammonium, methylamine, dimethylamine, trimethylamine, triethylamine, ethylamine, and the like.

Convenient modes of administration of compounds of the invention include parenteral (for example, subcutaneous, intravenous, intramuscular, intradermal, intraperitoneal, intrathecal, intraocular, intranasal, intraventricular injection or infusion techniques), oral, pulmonary (e.g. inhalation), transdermal application, topical (e.g. creams or gels or powders), or rectal administration. Depending on the route of administration, the formulation and/or compound may be coated with a material to protect the compound from the action of enzymes, acids and other natural conditions which may inactivate the therapeutic activity of the compound.

Dispersions of the compounds according to the invention may also be prepared in glycerol, liquid polyethylene glycols, and mixtures thereof and in oils. Under ordinary conditions of storage and use, pharmaceutical preparations may contain a preservative to prevent the growth of microorganisms.

Pharmaceutical compositions suitable for injection include: sterile aqueous solutions (where water soluble), or dispersions and sterile powders for the extemporaneous preparation of sterile injectable solutions or dispersions. Ideally, the composition is stable under the conditions of manufacture and storage and may include a preservative to stabilise the composition against the contaminating action of microorganisms such as bacteria and fungi.

In one embodiment of the invention, the compound(s) of the invention may be administered orally, for example, with an inert diluent or an assimilable edible carrier. The compound(s) and other ingredients may also be enclosed in a hard or soft shell gelatin capsule, compressed into tablets, or incorporated directly into an individual's diet. For oral therapeutic administration, the compound(s) may be incorporated with excipients and used in the form of ingestible tablets, buccal tablets, troches, capsules, elixirs, suspensions, syrups, wafers, and the like. Suitably, such compositions and preparations may contain at least 1% by weight of active compound.

The percentage of the compound(s) of the invention in pharmaceutical compositions and preparations may, of course, be varied. For example, the amount may conveniently range from about 2% to about 90%, about 5% to about 80%, about 10% to about 75%, about 15% to about 65%; about 20% to about 60%, about 25% to about 50%, about 30% to about 45%, or about 35% to about 45%, of the weight of the dosage unit. The amount of compound in therapeutically useful compositions is such that a suitable dosage can be obtained. Suitable dosages may be obtained by single or multiple administrations.

The term “pharmaceutically acceptable carrier” is intended to include solvents, dispersion media, coatings, anti-bacterial and anti-fungal agents, isotonic and absorption delaying agents, and the like. The use of such media and agents for pharmaceutically active substances is well known in the art. Except insofar as any conventional media or agent is incompatible with the compound, use thereof in the therapeutic compositions and methods of treatment and prophylaxis is contemplated. Supplementary active compounds may also be incorporated into the compositions according to the present invention. It is especially advantageous to formulate parenteral compositions in dosage unit form for ease of administration and uniformity of dosage.

The term “dosage unit form” as used herein refers to physically discrete units suited as unitary dosages for the individual to be treated; each unit containing a predetermined quantity of compound(s) calculated to produce the desired therapeutic effect in association with the required pharmaceutical earner. The compound(s) may be formulated for convenient and effective administration in effective amounts with a suitable pharmaceutically acceptable carrier in an acceptable dosage unit. In the case of compositions containing supplementary active ingredients, the dosages are determined by reference to the usual dose and manner of administration of the said ingredients.

In one embodiment, the carrier is an orally administrable carrier. In another embodiment, the carrier is suitable for intravenous administration. Another suitable form of the pharmaceutical composition is a dosage form formulated as enterically coated granules, tablets or capsules suitable for oral administration.

In a preferred embodiment, the compound(s) of the invention may be administered by injection. In the case of injectable solutions, the carrier may be a solvent or dispersion medium containing, for example, water (eg, water-for-injection), saline, 5% glucose solution, ethanol, polyol (for example, glycerol, propylene glycol, and liquid polyetheylene glycol, and the like), suitable mixtures thereof, and vegetable oils. The proper fluidity can be maintained, for example, by the use of a coating such as lecithin, by the maintenance of the required particle size in the case of dispersion and by the use of surfactants (eg, polysorbate 80). Prevention of the action of microorganisms can be achieved by including various anti-bacterial and/or anti-fungal agents. Suitable agents are well known to those skilled in the art and include, for example, parabens, chlorobutanol, phenol, benzyl alcohol, ascorbic acid, thimerosal, and the like. In many cases, it may be preferable to include isotonic agents, for example, sugars, polyalcohols such as mannitol, sorbitol, sodium chloride in the composition. Prolonged absorption of the injectable compositions can be brought about by including in the composition an agent which delays absorption, for example, aluminium monostearate and gelatin.

Sterile injectable solutions can be prepared by incorporating the analogue in the required amount in an appropriate solvent with one or a combination of the ingredients enumerated above, as required, followed by filtered sterilisation. Generally, dispersions are prepared by incorporating the analogue into a sterile vehicle which contains a basic dispersion medium and the required other ingredients from those enumerated above.

Tablets, troches, pills, capsules and the like can also contain the following: a binder such as gum gragacanth, acacia, corn starch or gelatin; excipients such as dicalcium phosphate; a disintegrating agent such as corn starch, potato starch, alginic acid and the like; a lubricant such as magnesium stearate; and a sweetening agent such as sucrose, lactose or saccharin or a flavouring agent such as peppermint, oil of wintergreen, or cherry flavouring. When the dosage unit form is a capsule, it can contain, in addition to materials of the above type, a liquid carrier. Various other materials can be present as coatings or to otherwise modify the physical form of the dosage unit. For instance, tablets, pills, or capsules can be coated with shellac, sugar or both. A syrup or elixir can contain the analogue, sucrose as a sweetening agent, methyl and propylparabens as preservatives, a dye and flavouring such as cherry or orange flavour. Of course, any material used in preparing any dosage unit form should be pharmaceutically pure and substantially non-toxic in the amounts employed. In addition, the analogue can be incorporated into sustained-release preparations and formulations.

Preferably, the pharmaceutical composition may further include a suitable buffer to minimise acid hydrolysis. Suitable buffer agent agents are well known to those skilled in the art and include, but are not limited to, phosphates, citrates, carbonates and mixtures thereof.

Single or multiple administrations of the pharmaceutical compositions according to the invention may be carried out.

Generally, an effective dosage per 24 hours may be in the range of about 0.0001 mg to about 1000 mg per kg body weight (mg/kg); suitably, about 0.001 to about 750 mg/kg body weight; about 0.01 to about 500 mg/kg; about 0.1 to about 500 mg/kg; about 0.1 to about 250 mg/kg body weight; or about 1.0 to about 250 mg/kg. Still suitably, an effective dosage per 24 hours may be in the range of about 1.0 to about 200 mg/kg; about 1.0 to about 100 mg/kg body weight; about 1.0 to about 50 mg/kg; about 1.0 to about 25 mg/kg; about 5.0 to about 50 mg/kg; about 5.0 to about 20 mg/kg; or about 5.0 to about 15 mg/kg.

Alternatively, an effective dosage may be calculated according to the Body Surface Area (BSA) of the patient to be treated. The BSA of a patient may be readily calculated using methods known to those skilled in the art. A suitable dose generally may be up to about 500 mg/m². For example, generally, an effective dosage may be in the range of about 10 to about 500 mg/m², about 25 to about 350 mg/m², about 25 to about 300 mg/m², about 25 to about 250 mg/m², about 50 to about 250 mg/m², and about 75 to about 150 mg/m².

By way of example, suitable dosage forms in accordance with the present invention include the following:

### Tablet

| Compounds of the invention | 0.01 to 20 mg, generally 0.1 to 10 mg | |
|---|---|---|
| Starch | 10 to 20 | mg |
| Lactose | 100 to 250 | mg |
| Gelatin | 0 to 5 | mg |
| Magnesium Stearate | 0 to 5 | mg |

### Injectable Solution

| Compounds of the invention | 0.01 to 20 mg, generally 0.1 to 10 mg | |
|---|---|---|
| Sodium Chloride | 8.5 | mg |
| Potassium Chloride | 3 | mg |
| Calcium Chloride | 4.8 | mg |
| Water for injection, q.s. to | 10 | ml |

It will be appreciated by a person skilled in the art that numerous variations and/or modifications may be made to the invention as shown in the specific embodiments without departing from the spirit or scope of the invention as broadly described. The present embodiments are, therefore, to be considered in all respects as illustrative and not restrictive.

### EXAMPLES

### Materials and Methods

### Chemicals

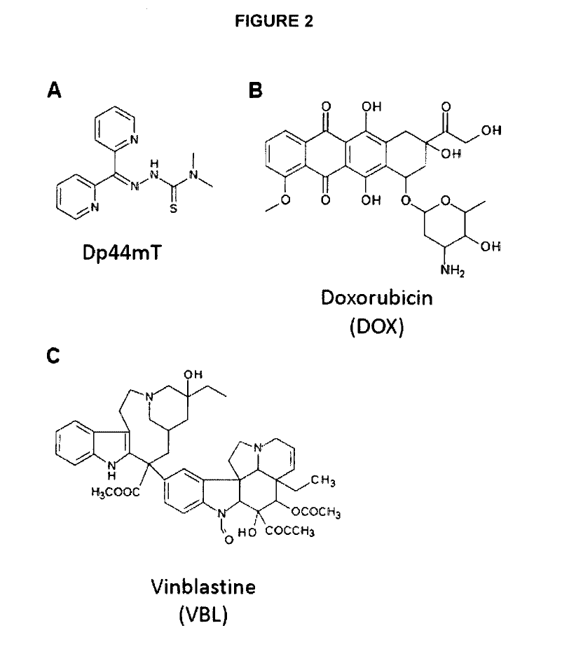

Doxorubicin (DOX) was purchased from Pfizer (New York City, N.Y.). Vinblastine (VBL), paclitaxel (PAC), methylamine (MA), ammonium chloride (NH₄Cl), copper(II) chloride (CuCl₂), Rhodamine 123 (Rh123), tetrathiolmolybdate (TM) and chloroquine (CLQ) were purchased from Sigma-Aldrich (St Louis, Mo.). Valspodar (Val; PSC833) was kindly provided by Novartis (Basil, Switzerland). Elacridar (Ela; GF120918) was a gift from GlaxoSmithKline (London, UK). Lysotracker® red was purchased from Life Technologies (Carlsbad, Calif.). ¹⁴C-Doxorubicin hydrochloride was obtained from Perkin-Elmer (Waltham, Mass.). Both Dp44mT (FIG. 2A), the copper(II) complex of Dp44mT (Cu[Dp44mT]) and 2-benzoylpyridine-4-ethyl-3-thiosemicarbazone was synthesized and characterized as previously described.^{9,19,20} Lipofectamine 2000 was purchased from Life Technologies. The ¹⁴C-Dp44mT was custom prepared by The Institute of Isotopes Ltd. (Budapest, Hungary).

### Cell Culture

Human cervical carcinoma derived KB-3-1 (KB31) cells and the VBL-resistant variant KB-V-1 (KBV1) cells (grown in 1 μg/mL VBL) were a kind gift from Dr. M. Kavallaris (Children's Cancer Institute Australia, Sydney, Australia). The 2008 ovarian carcinoma cell line and the PAC-resistant 2008/P200A cell line (grown in 200 ng/mL of PAC) were kindly provided by Dr. John Allen (Centenary Institute, Sydney, Australia). Cells were grown in Dulbecco's modified Eagle's medium (DMEM; Life Technologies) with 10% (v/v) fetal calf serum (Sigma-Aldrich, St. Louis, Mo.) and supplemented with the following additions from Life Technologies: 1% (v/v) sodium pyruvate, 1% (v/v) non-essential amino acids, 100 U/mL penicillin, 100 μg/mL streptomycin, 2 mM L-glutamine and 0.28 μg/mL fungizone. The cells were then incubated at 37° C. in a humidified atmosphere of 5% CO₂ and 95% air.

### MTT Proliferation Assay

The effect of anti-cancer agents on cellular proliferation was examined using the MTT [3-(4,5-dimethylthiazol-2-yl)-2,5-diphenyl tetrazolium] method. In this assay, 3×10³ cells/well were seeded in 96-well plates in 100 μL of media. The next day, serially diluted drugs of interest were added. In those experiments using Pgp inhibitors, Ela and Val at 0.1 μM and 1 μM were used, respectively. In studies implementing the lysosomotropic agents, only concentrations that did not affect cellular viability over a period of 72 h were used, namely NH₄Cl (3 mM), MA (100 μM) and CLQ (1 μM). The cells were incubated at 37° C. in a humidified atmosphere containing 5% CO₂ and 95% air for 72 h. After this incubation, 10 μL of MIT (5 mg/mL) was added to each well and the incubation continued at 37° C. for 2 h. After solubilization of the cells with 100 μL of 10% SDS-50% isobutanol in 10 mM HCl, the plates were read at 570 nm using a scanning multi-well spectrophotometer (Victor™ Multilabel Counter plate reader; Perkin Elmer, Australia). Results were analyzed to calculate the concentration of chelator or complex necessary to reduce the absorbance to 50% (IC₅₀) of the control. Using this method, absorbance was shown to be directly proportional to cell counts.

Transient Knockdown of Pgp (MORI) Using siRNA

For all siRNA treatments against MDR1 (MDR1-siRNA; Cat #: 4123, Life Technologies), a siRNAlipofectamine mixture (50 nM MDR1 siRNA and 1/400 lipofectamine 2000) was added to the cells (at 30% confluency) and incubated for 72 h/37° C. prior to further experimentation. The effectiveness of Pgp knockdown was confirmed using western blot and also assessing DOX cytotoxicity via the MTT assay. As a control, scrambled siRNA (Scr siRNA; Life Technologies) was used at the same concentration as MDR1 siRNA.

### Western Blot

Standard methods were used for western blot21 using an antibody against Pgp (Cat. #; P7965 Clone: F4; Sigma-Aldrich). Blots were also probed for β-actin as a protein loading control (Sigma-Aldrich).

### Pgp-ATPase Activity Assay

The ATPase activities of Pgp were determined using Pgp-enriched membranes and a luminescent ATP detection kit (Pgp-Glo Assay Kit, Promega, USA) according to the manufacturer's instructions. Briefly, Pgp membranes (0.5 mg/mL) and Mg(II)-ATP (5 mM) were incubated in the absence or presence of sodium orthovanadate (100 μM) for 40 min/37° C. and ATP detected as a luciferase-generated luminescent signal. Basal Pgp-ATPase activities were determined as the difference between the ATP hydrolysis in the presence or absence of sodium orthovanadate. Verapamil-stimulated Pgp-ATPase activity was measured in the presence of the Pgp control substrate, verapamil (200 μM).

Test substrates (Dp44mT and Bp4eT) of interest were dissolved in dimethyl sulfoxide. The final concentration of DMSO in the assay medium was less than 1%. Control experiments indicated that DMSO at this concentration had no effect on ATPase activity.

### Uptake/Efflux of ¹⁴C-Doxorubicin, ³H-Vinblastine and ¹⁴C-Dp44mT

For the uptake assay, each 35 mm culture dish containing 5×10⁵ cells was incubated with ¹⁴C-doxorubicin, ³H-vinblastine or ¹⁴C-Dp44mT (all at 1 μCi) for 30 min/37° C. Cells were then washed on ice three times with ice-cold PBS and collected for analysis. Drug uptake was calculated as the number of drug molecules/cell. In experiments using Pgp inhibitors, prior to radio-labeled drug treatment, the cells were pre-incubated with Val (1 μM) or Ela (0.1 μM) for 30 min/37° C. For studies using lysosomotropic agents, cells were pre-incubated with NH₄Cl (5 mM), CLQ (100 μM) or MA (100 μM) for 30 min/37° C. prior to ¹⁴C-Dp44mT uptake. Notably, the concentration of lysosomotropic agents used for these studies are higher than those used in MTT experiments due to the very short incubation periods used and did not cause cytotoxicity. For efflux assays, the same labeling procedure was conducted as in the uptake assay. The cells were then washed three times on ice with ice-cold PBS and then reincubated up to 10 min/37° C. with media alone or media containing Val (1 μM) or Ela (0.1 μM). After this procedure, the overlying media was removed using a Pasteur pipette and then placed in a counting vial. The cells were removed from the plate in 1 mL of PBS and then dispensed into a separate counting vial. The radiolabeled-drug efflux was calculated as a percentage of the total drug uptake by the cells found at zero time.

### Rhodamine 123 Accumulation Assay

For the Rh123 accumulation assay, cells were pre-incubated with a range of concentrations (0.001-10 μM) of Dp44mT, Val or Ela for 30 min/37° C., followed by incubation with Rh123 (1 μg/mL) for 15 min/37° C. and subsequently washed on ice with ice-cold PBS. Cells were then harvested and resuspended in ice-cold PBS and kept on ice until flow cytometric analysis (see below).

### Assessment of Lysosomal Membrane Permeability

Lysotracker® red (Life Technologies) and acridine orange (Sigma-Aldrich) are dyes showing high specificity for lysosomes and were used to determine lysosomal membrane permeability (LMP). The Lysotracker® red was visually assessed by fluorescence microscopy utilizing a Zeiss Axio Observer.Z1 Fluorescence Microscope equipped with an AxioCam camera (Zeiss, Oberkochen, Germany) and acridine orange was quantified by flow cytometry (see below).

Briefly, cells were incubated with Lysotracker® red (50 nM) for 15 min/37° C. or acridine orange (20 nM) for 15 min/37° C., washed on ice 3 times with ice-cold PBS and then incubated with Cu[Dp44mT] (25 μM) for 30 min/37° C. or Dp44mT alone (25 μM) for 30 min or 24 h/37° C.

### Flow Cytometry

The dyes, Rh123 and acridine orange were detected with the FACS Canto flow cytometer (BD Biosciences) and 10,000 events were acquired for every sample. Data analysis was performed using FlowJo software, version 7.5.5 (Tree Star Inc., Ashland, OR).

### Redox Studies: Oxidation of H2DCF

The inhibitory effect of Val, Ela or TM (all at 5 μM) on ROS generation by Cu[Dp44mT] was examined using H2DCF (5 μM) in the absence of cells over a 12 min time period using standard methods. These studies were conducted in an acetate buffer (150 mM; pH 5) containing cysteine (100 μM) to mimic lysosomal conditions. In these experiments, hydrogen peroxide (100 μM) was added to initiate ROS generation. To confirm ROS production, DMSO (10% v/v) was used, as it has been shown to be an effective hydroxyl radical scavenger.

### Statistics

Data were compared using the Student's t-test. Results were expressed as mean±SD (number of experiments) and considered to be statistically significant when p<0.05.

### Example 1

### A) Thiosemicarbazones Dp44mT and Bp4eT Exhibit Increased Cytotoxicity to Cells in the Presence of Functional Pgp

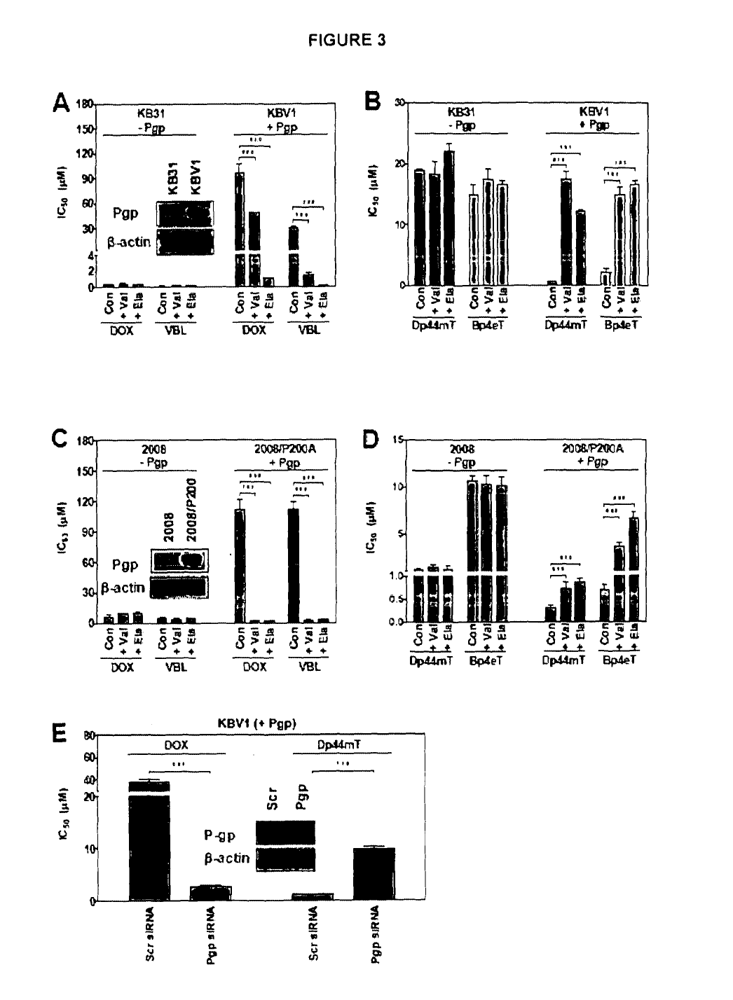

We have previously reported that the thiosemicarbazone, Dp44mT, exhibits increased cytotoxicity to Pgp over-expressing KBV1 cells relative to KB31 cells that do not express Pgp.⁵ However, whether this was related to the expression of Pgp was not established. To determine that the potentiated cytotoxicity of Dp44mT20 is in fact Pgp-dependent, two pairs of well characterized Pgp-expressing and their non-Pgp expressing counterpart cells (KB31 cells vs. KBV1^{6,7} and 2008⁸ vs. 2008/P200A) were used in this study (FIG. 3A-E). In addition, another related thiosemicarbazone, Bp4eT,⁹ was assessed to see if potentiated cytotoxic activity was also observed.

In initial studies, the expression of Pgp was examined by western blot in both pairs of cell lines and shown to be marked in KBV1 and 2008/P200 cells, while being undetectable in KB31 and 2008 cells (FIG. 3A, C). As an important positive control, after a 72 h incubation with the well characterized chemotherapeutic Pgp substrates, DOX and VBL 41, the drug-resistant KBV1 cells showed 220-fold and 221-fold increase in resistance for DOX (IC₅₀: 96.4±10.1 μM) and VBL (IC₅₀: 29.7±1.77 μM) compared to KB31 cells (IC₅₀: 0.44±0.003 μM; IC₅₀: 0.13±0.004 μM p<0.001) respectively (FIG. 3A). Significantly, Pgp was confirmed to be responsible for the increase in MDR by DOX and VBL as the resistance in KBV1 cells to DOX and VBL were markedly sensitized in the presence of the potent Pgp inhibitors, Val and Ela (FIG. 3A). Moreover, no significant (p>0.05) change in cytotoxicity of DOX and VBL could be observed in non-Pgp expressing KB31 cells when treated with Val and Ela (FIG. 3A).

Interestingly, in marked contrast to DOX and VBL, the thiosemicarbazones Dp44mT and Bp4eT were 31-fold and 6.7-fold more cytotoxic (p<0.001) in drug-resistant KBV1 cells compared to KB31 cells, respectively (FIG. 3B). In order to investigate if the potentiated sensitivity observed in KBV1 cells by Dp44mT and Bp4eT was a Pgp-dependent effect, the Pgp inhibitors, Val or Ela, were co-incubated with Dp44mT and Bp4eT. Again, in contrast to the DOX and VBL treatment, Pgp inhibition led to a clear decrease in the cytotoxicity (increase in the IC₅₀ value) of Dp44mT and Bp4eT in KBV1 cells, suggesting that the sensitivity observed with these thiosemicarbazones was Pgp-dependent (FIG. 3B). In agreement with this, Pgp inhibition by Val and Ela did not significantly alter their cytotoxicity in non-Pgp expressing KB31 cells (FIG. 2B, 3B).

Similar results were obtained in another set of cell line pairs expressing and not expressing Pgp (FIG. 3C, D). Indeed, the non-Pgp expressing 2008 cells were extremely sensitive to DOX and VBL compared to Pgp expressing 2008/P200A cells which demonstrated significantly higher resistance towards DOX and VBL (see Controls; FIG. 3C). While the Pgp inhibitors, Val and Ela, abrogated the resistance against DOX and VBL observed in 2008/P200A cells, the inhibitors did not significantly (p>0.05) affect the cytotoxicity of DOX and VBL in non-Pgp expressing 2008 cells (FIG. 3C). Similar to KBV1 cells, Pgp-dependent sensitivity of Dp44mT and Bp4eT was demonstrated in the Pgp overexpressing 2008/P200A cells relative to non-Pgp-expressing 2008 cells (cf controls in FIG. 3D). In fact, as observed with KBV1 cells, 2008/P200A showed significantly (p<0.001) increased sensitivity to Dp44mT and Bp4eT compared to non-Pgp expressing 2008 cells (FIG. 2D, 3D). Co-incubation with Val or Ela again significantly (p<0.001) decreased sensitivity of Dp44mT and Bp4eT to Pgp-expressing 2008/P200A cells, but no significant (p>0.05) change was observed in the presence of these inhibitors in non-Pgp expressing 2008 cells (FIG. 2D, 3D). Together, these results demonstrate that the increased sensitivity of cells to the cytotoxic activity of Dp44mT and Bp4eT is Pgp-dependent.

### B) Knockdown of Pgp (MDR1) by siRNA Decreases Sensitivity to Dp44mT

To further establish that the increased sensitivity observed for Dp44mT is mediated by Pgp, gene silencing of Pgp was employed using siRNA to inhibit MDR1 mRNA which is translated to Pgp protein. Gene silencing was achieved by transfection of a synthetic siRNA against the MDR1 gene (Pgp siRNA) and the effect of down-regulation was assayed 72 h after transfection. The Pgp protein level was reduced by 85±3.3% (see insert in FIG. 3E) in KBV1 cells treated with Pgp siRNA, compared to cells transfected with a scrambled siRNA (Pgp siRNA vs. Scr siRNA; FIG. 3E). After treatment with either Scr siRNA or siRNA targeted against Pgp, cytotoxicity of DOX or Dp44mT was measured after a 72 h incubation (FIG. 3E). While the knockdown of Pgp significantly (p<0.001) decreased the IC₅₀ of DOX by 15-fold, the IC₅₀ of Dp44mT was increased by 10-fold (FIG. 2E). Collectively, these results signify the requirement of Pgp in the potentiated toxicity exerted by Dp44mT.

### C) Dp44mT is a Pgp Substrate

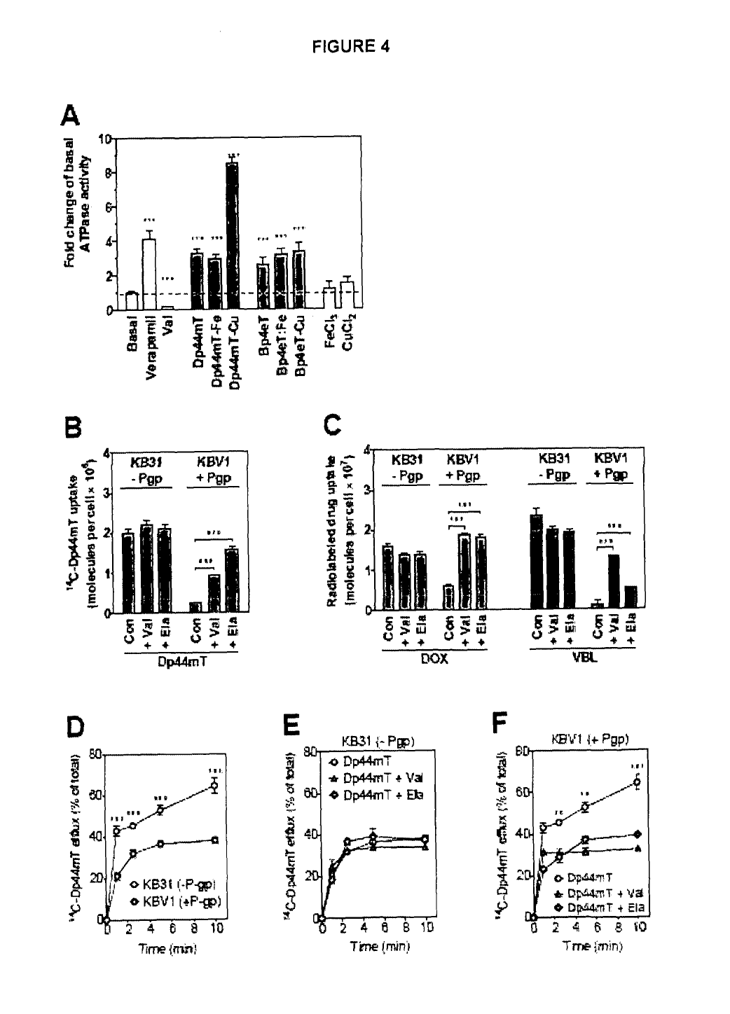

Since expression and function of Pgp is a pre-requisite for the increased cytotoxicity of Dp44mT (FIG. 3B, D, E), we evaluated the potential interaction between Pgp and Dp44mT (FIG. 4A-F). The Pgp Glo™ ATPase assay (Promega, Madison, Wis.) was used to assess if Dp44mT and Bp4eT are substrates of Pgp. The ATPase assay measures the Pgp-mediated ATPase activity of Pgp by quantitating ATP consumption in the presence of the compounds of interest after incubation with purified membrane proteins containing high levels of Pgp. The ATPase activity of Pgp is stimulated in the presence of transported substrates, such as verapamil, and this activation is prevented by potent Pgp inhibitors, such as Val. The basal ATPase activity of Pgp is compared to the ATPase activity stimulated or inhibited by the compounds of interest. If the ATPase activity is above the basal activity, it is considered to activate ATPase activity as a Pgp substrate, while less than the basal activity suggests that it is an inhibitor of Pgp-mediated ATPase activity. Similar to verapamil, Dp44mT and its respective Fe and Cu complexes significantly (p<0.001) stimulated the basal catalytic activity by ˜3- to 8-fold (FIG. 3A, 4A). Interestingly, the Dp44mT-Cu complex demonstrated the highest ATPase activity, while the Dp44mT-Fe complex had a similar level of activity as that of the Dp44mT ligand alone. In contrast, while Bp4eT also demonstrated significant (p<0.001) stimulation of ATPase activity, its Fe and Cu complexes demonstrated similar activity as the ligand alone. The Pgp inhibitor, Val significantly (p<0.001) prevented the stimulation of the ATPase, decreasing the ATPase activity by 5-fold relative to the basal control (FIG. 4A). Neither FeCl₃ nor CuCl₂ significantly stimulated ATPase activity and acted as negative controls. These results indicated that both the thiosemicarbazones Dp44mT and Bp4eT interact with Pgp and lead to activation of the Pgp-mediated ATPase (FIG. 4A). Since the cytotoxicity assays (FIG. 3B, 3D) and Pgp-ATPase activity (FIG. 4A) showed somewhat similar results for both Bp4eT and Dp44mT, it was decided to focus on Dp44mT for future studies.

To further verify the interaction of Dp44mT with Pgp, radio-labeled compounds were used to determine their differential uptake and efflux in Pgp-expressing KBV1 and non-expressing KB31 cells (FIG. 4B-F) Notably, in these studies, ¹⁴C-DOX and ³H-VBL were used as positive controls as they are well characterized Pgp substrates relative to the test compound, ¹⁴C-Dp44mT. Cellular accumulation of ¹⁴C-Dp44mT, ¹⁴C-DOX and ³H-VBL was significantly (p<0.001) less in the control groups in Pgp expressing KBV1 cells relative to KB31 cells (FIG. 4B, 4C). Then, Pgp inhibitors were examined to assess if the reduced accumulation of the radiolabels above was indeed Pgp-dependent. The results showed that the Pgp inhibitors, Val and Ela, significantly (p<0.001) increased the uptake of ¹⁴C-Dp44mT, ¹⁴C-DOX, and ³H-VBL in Pgp-expressing KBV1 cells versus the relative control, while these inhibitors did not significantly alter radiolabel uptake in non-Pgp expressing KB31 cells versus the relative control (FIG. 4B, 4C). These observations suggest that in addition to ¹⁴C-DOX and ³H-VBL, ¹⁴C-Dp44mT is also a substrate of Pgp. This was further supported by efflux studies using radio-labeled ¹⁴C-Dp44mT where significantly (p<0.001) higher efflux of ¹⁴C-Dp44mT in Pgp over-expressing KBV1 cells was observed in comparison to KB31 cells after a 1-10 min incubation (FIG. 3D).

This efflux was a Pgp-dependent effect as the inhibitors, Val and Ela, significantly (p<0.01-0.001) suppressed efflux only in Pgp over-expressing KBV1 cells, but not in KB31 cells (cf. FIGS. 4E and 4F). Taken together, the reduced uptake, increased efflux and the Pgp-dependent ATPase activity observed demonstrate that Dp44mT is a substrate of Pgp.

### D) Dp44mT is an Inhibitor of Pgp

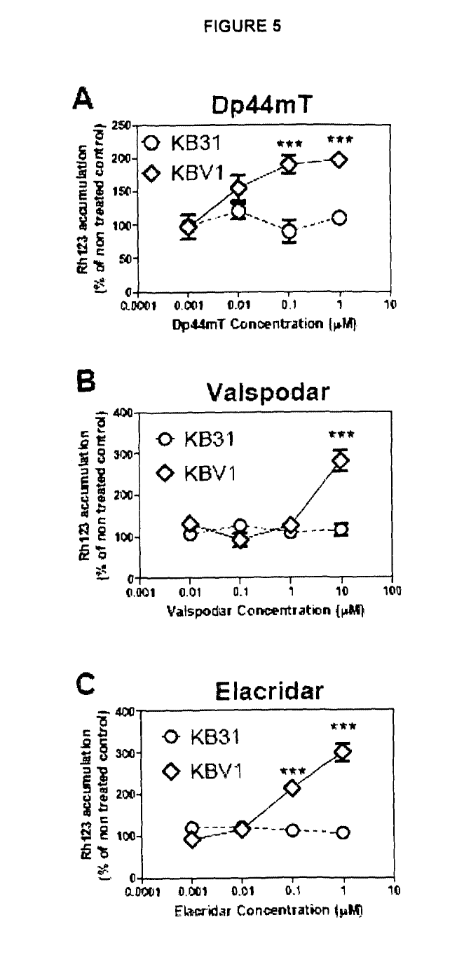

Since Pgp substrates such as verapamil have also been demonstrated to be competitive inhibitors of Pgp at higher concentrations, we next assessed if Dp44mT can also act as an inhibitor of Pgp. To study this, the accumulation of the well characterized fluorescent Pgp substrate, Rh123, was examined. If Dp44mT is a Pgp inhibitor, an increase in cellular Rh123 accumulation would be observed as the concentration of Dp44mT is increased. The results showed that as the concentration of Dp44mT increased to 0.1 μM or above, there was a significantly (p<0.001) increased accumulation of Rh123 by up to two fold in Pgp over-expressing KBV1 cells (FIG. 5A). In contrast, increased concentrations of Dp44mT did not significantly affect Rh123 accumulation in non-Pgp expressing KB31 cells (FIG. 5A). The potent Pgp inhibitors, Val (FIG. 5B) and Ela (FIG. 5C), were used as positive controls for inhibiting Pgp and showed a significant (p<0.001) 3-fold increase in Rh123 retention as a function of inhibitor concentration in KBV1 cells at 10 and 0.1 μM, respectively (FIG. 5B-C) As for Dp44mT, the inhibitors had no significant effect on Rh123 accumulation in KB31 cells (FIG. 5B-C).

The higher potency of Ela compared to Val in terms of inhibiting Pgp activity is consistent with the affinity of these agents for Pgp. The Rh123 accumulation studies (FIG. 5A), the Pgp Glo™ ATPase assay (FIG. 4A) and uptake/efflux studies with ¹⁴C-Dp44mT (FIG. 4B-F)demonstrated that Dp44mT acts similarly as verapamil by being a substrate itself and also an inhibitor of Pgp.

### E) Dp44mT is Sequestered into Lysosomes in a Pgp-Dependent Manner

A previous investigation demonstrated that functional lysosomal Pgp in KBV1 and 2008/P200 cells traps Pgp substrates such as DOX in the lysosome. In fact, if Pgp substrates are charged at acidic pH this will decrease their permeability through the lysosomal membrane leading to lysosomal trapping. Considering that Dp44mT is a substrate of Pgp (FIG. 4A), and also that it can become charged at a lysosomal pH of 5, we examined if Pgp can increase the uptake and trapping of ¹⁴C-Dp44mT in lysosomes. In order to test this hypothesis, the well characterized lysosomotropic agents NH₄Cl, MA or CLQ (that increase lysosomal pH) were used to prevent trapping of ¹⁴C-Dp44mT in lysosomes by neutralizing its charged species and allowing transport across the lysosomal membrane.

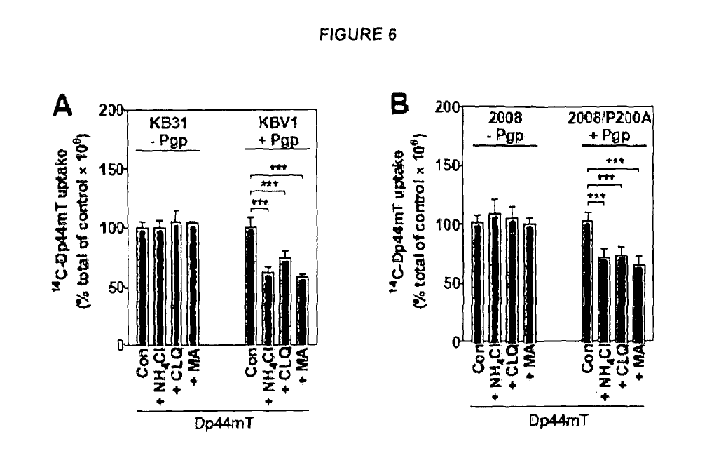

Pre-treatment of cells with each lysosomotropic agent significantly (p<0.001) decreased ¹⁴C-Dp44mT accumulation in Pgp over-expressing KBV1 (FIG. 6A) and 2008/P200 cells (FIG. 6B). Importantly, all three lysosomotropic agents did not significantly affect ¹⁴C-Dp44mT accumulation in non-Pgp expressing KB31 (FIG. 6A) or 2008 (FIG. 6B) cells. These results suggest that lysosomotropic agents decrease Pgp-dependent ¹⁴C-Dp44mT accumulation in the lysosomal compartment.

### F) Dp44mT Potentiates Lysosomal Damage in Pgp Over-Expressing Cells

Previously, we reported that Dp44mT targets lysosomes and after copper binding and the generation of cytotoxic reactive oxygen species this leads to lysosomal membrane permeabilization. Considering our studies above demonstrating that Pgp actively sequesters Dp44mT into lysosomes (FIG. 6A, B), the effect on lysosomal damage was then studied by examining the release of the classical lysosomal marker, Lysotracker® red, from lysosomes of KBV1 cells. The Cu[Dp44mT] complex was used as we previously showed: (i) that it caused very rapid permeabilization of lysosomes; (ii) it was found to be a Pgp substrate (FIG. 4A); and (iii) formed spontaneously when Dp44mT enters cells and chelates intracellular copper.

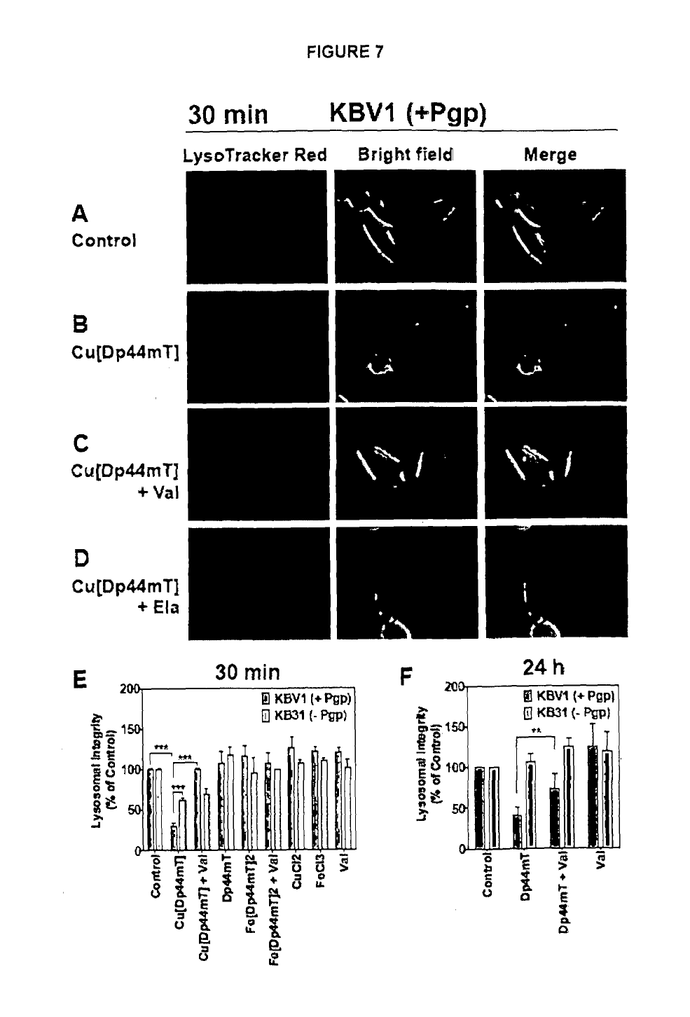

Results using Pgp-positive KBV1 cells labeled with Lysotracker® red showed that under control conditions, the classical particulate lysosomal pattern in the cytosol was observed (FIG. 7A). However, upon incubation of these cells for 30 min with the Cu[Dp44mT] complex, the particulate staining with Lysotracker® red disappeared (FIG. 7B), which is consistent with lysosomal membrane permeabilization¹⁰ that was observed in our previous studies with this complex. The loss of Lysotracker® red staining induced by Cu[Dp44mT] was prevented by both Pgp inhibitors, namely Val and Ela (FIG. 7C, D).

To further examine if the lysosomal damage induced by the Cu[Dp44mT] complex was Pgp-dependent, lysosomal integrity was assessed using another classical lysosomal marker, acridine orange. A decrease in acridine orange intensity as measured by flow cytometric analysis is consistent with a loss of lysosomal integrity. In agreement with the microscopic studies using Lysotracker® red (FIG. 7B), a 30 min incubation with the Cu[Dp44mT] complex decreased lysosomal integrity to a significantly (p<0.001) greater extent in Pgp-expressing KBV1 cells compared to KB31 cells (FIG. 7E).

However, the Pgp inhibitor, Val, significantly (p<0.001) prevented the Cu[Dp44mT]-induced lysosomal damage in KBV1 cells, suggesting that Pgp function plays an important role in lysosomal damage by Cu[Dp44mT]. Subsequently, the ability of Dp44mT, Fe[Dp44mT]₂, CuCl₂, FeCl₃ or Val to cause lysosomal damage was also investigated and did not compromise lysosomal integrity after a 30 min incubation (FIG. 7E). However, after longer incubations of 24 h, Dp44mT decreased lysosomal integrity in KBV1 cells, but not in KB31 cells (FIG. 7E). The Pgp inhibitor, Val, significantly (p<0.01) reduced lysosomal damage by Dp44mT in KBV1 cells (FIG. 6, 7F), indicating that Pgp expression could induce increased lysosomal damage mediated by Dp44mT.

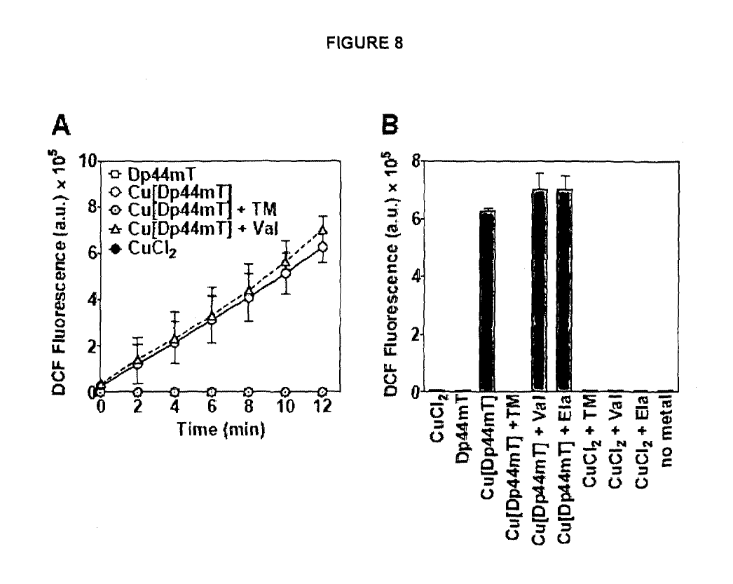

Since Cu[Dp44mT] was the species responsible for rapidly compromising lysosomal integrity (FIG. 7E), subsequent studies assessed if the Pgp inhibitors, Val and Ela, were preventing Cu[Dp44mT]-induced lysosomal damage by direct scavenging of ROS. The ROS scavenging ability of the inhibitors were assayed using a cell-free system under lysosomal-like conditions (FIGS. 8A and B). Oxidative stress was determined using the well-characterized probe, H₂DCF, for assessing redox stress. The Cu[Dp44mT] complex (5 μM) displayed high redox activity over 12 min, while Dp44mT and CuCl₂ showed no activity (FIG. 8A). Significantly, the redox activity of Cu[Dp44mT] was totally prevented by the Cu chelator, TM (5 μM; FIG. 8A, B), consistent with the ability of TM to bind Cu from Cu[Dp44mT]. On the other hand, Val (5 μM) was not able to prevent the redox activity of Cu[Dp44mT] over time (FIG. 8A). Similar to Val, Ela also did not alter ROS-generation induced by Cu[Dp44mT] (FIG. 7B). These results indicate that Val and Ela are not directly scavenging ROS under lysosomal-like conditions. Hence, the prevention of lysosomal damage by Val and Ela after Dp44mT and Cu[Dp44mT] treatment described above (FIG. 7) is probably due to inhibition of Pgp.

### Example 2

MDR is a major obstacle for successful treatment outcomes in cancer. The inventors and others have previously demonstrated that certain drugs can possess potentiated cytotoxicity in MDR cells relative to drug-sensitive cells. However, the molecular mechanism of how these agents precisely overcome MDR is still unknown. The inventors have shown that one of these drugs, namely Dp44mT, accumulates within lysosomes to compromise lysosomal membrane integrity and induce cell death. Moreover, it has also been demonstrated that MDR is conferred by Pgp localized not only on the plasma membrane, but also by Pgp within the lysosomal membrane which results in the sequestration of a Pgp substrate i.e., DOX into lysosomes.²¹ Considering this, the current study examined: (1) if the increased cytotoxicity of Dp44mT in MDR cells was Pgp-dependent; (2) whether Dp44mT was a Pgp substrate or an inhibitor, and (3) if there was a link between the potentiated cytotoxicity of Dp44mT in Pgp-expressing cells and lysosomal damage. For the first time, we demonstrate that Pgp substrates such as Dp44mT can “hijack” lysosomal Pgp to selectively overcome drug resistance by inducing increased lysosomal damage in Pgp-expressing cells. Hence, agents like Dp44mT offer a novel and exciting therapeutic strategy to combat MDR by targeting Pgp itself.

### A) Dp44mT Exerts Potentiated Cytotoxicity in MDR Cells Due to Pgp Expression

By using the Pgp inhibitors, Val and Ela, in KBV1 and 2008/P200A cells (FIG. 3B, D) and the selective knockdown of Pgp in KBV1 cells (FIG. 3E), it has been demonstrated that Pgp plays a crucial role in the potentiated cytotoxicity of Dp44mT in MDR cells. Both Pgp inhibition and down-regulation of Pgp in KBV1 cells led to a marked decrease in the sensitivity to Dp44mT. This was in contrast to the classical Pgp substrates, DOX and VBL, where Pgp inhibitors increased the sensitivity to these agents in Pgp-expressing KBV1 and 2008/P200A cells (FIG. 3A, C). Collectively, these results demonstrate that Pgp expression and function are critical to the potentiated cytotoxicity reported for Dp44mT.

### B) Dp44mT is a Substrate and Inhibitor of Pgp

Similarly to Dp44mT, another thiosemicarbazone compound, namely NSC73306, has been reported by others to demonstrate increased cytotoxicity to Pgp-expressing cells, although the exact molecular mechanism was not clear. Although enhanced cytotoxicity to NSC73306 required functional Pgp expression, biochemical assays revealed no direct interaction between NSC73306 and Pgp. However, since other agents that demonstrate increased cytotoxicity in Pgp-expressing cells have been reported to be Pgp substrates or inhibitors, the inventors assessed the direct interaction of Dp44mT with Pgp by utilizing assays: (1) Pgp ATPase activity when the Dp44mT interacted with isolated membrane Pgp (FIG. 4A); (2) ¹⁴C-Dp44mT efflux/uptake in the presence and absence of Pgp inhibitors (FIG. 4B, D-F); and (3) accumulation of the fluorescent Pgp substrate Rh123 in the presence and absence of Dp44mT (FIG. 5A). All of these assays demonstrate that Dp44mT interacts with Pgp, and in contrast to NSC73306, Dp44mT stimulates Pgp-ATPase activity, suggesting that Dp44mT is a Pgp substrate. The reported lack of a direct interaction between NSC73306 and Pgp despite increased sensitivity to Pgp-expressing cells, has been suggested to be because NSC73306 metabolites are the active form of this agent in cancer cells. In addition to the interaction of Dp44mT with Pgp, this agent was also found to be an inhibitor of this protein since it acted similarly to Val and Ela when added at high concentrations to Pgp-expressing cells only (FIG. 5A-C). Indeed, in these experiments, high Dp44mT levels led to accumulation of the fluorescent Pgp substrate, Rh123, in Pgp-expressing KBV1 cells. Collectively, the current studies demonstrate for the first time that Dp44mT is a substrate and inhibitor of Pgp and this may play role in its ability to potentiate cytotoxicity in Pgp-expressing cells.

### C) Hijacking of Lysosomal Pgp by Dp44mT Increases Cytotoxicity Via Lysosomal Damage

Along with Dp44mT and NSC73306, there have been other agents such as the lanthanum trisphenathroline complex KP772 that exhibits increased sensitivity to Pgp-expressing cells compared to parent cells. However, the molecular mechanism underlying the increased sensitivity observed with any of these cytotoxics has not been elucidated. The induction of ROS by these agents has been suggested to be linked to the potentiated cytotoxicity in MDR cells. Both NSC73306 and Dp44mT are chelators that bind intracellular metals which can cycle between two redox states^{11,12} and generate ROS.¹³ However, ROS generation alone by these agents cannot explain the mechanism behind the potentiated cytotoxicity observed in Pgp-expressing cells, as both MDR cells and their sensitive counterparts should be equally affected by ROS.¹⁴

The present inventors have shown that Dp44mT accumulates in lysosomes due to it becoming charged and membrane impermeable within the acidic environment (pH 5) of this organelle.¹⁵ Furthermore, the ability of Dp44mT to form a copper complex that generates cytotoxic ROS is responsible for inducing lysosomal permeabilization and cell death. The present inventors have demonstrated that the cytotoxic drug, DOX, which is also a Pgp substrate, can be actively transported into lysosomes via lysosomal Pgp, leading to its accumulation within this organelle.²¹ The accumulation was again because DOX becomes positively charged within the acidic environment of the lysosome, inhibiting its transport out of this organelle. Significantly, it has been shown that the addition of lysosomotropic agents prevented DOX accumulation in the lysosome and led to its delocalization to the cytosol. Therefore, the present inventors investigated if lysosomal Pgp increased the accumulation of ROS-generating agents such as Dp44mT into lysosomes, resulting in more damage to MDR cells than their non-resistant counterparts. Since Dp44mT can be charged and trapped in the acidic environment of lysosomes, raising the lysosomal pH with lysosomotropic agents could also increase the proportion of the neutral species of the drug preventing its trapping in lysosomes. The neutral species of Dp44mT will then be able to transverse the lysosomal membrane and exit the cell. Indeed, incubation of ¹⁴C-Dp44mT with three different lysosomotropic agents¹⁶ decreased ¹⁴C-Dp44mT uptake only in Pgp-expressing cells. These observations suggest that, as previously found for DOX, lysosomal Pgp can facilitate transport and accumulation of Dp44mT in lysosomes and that lysosomotropic agents can prevent this (FIG. 6A, B).

Studies examining lysosomal integrity utilizing the classical lysosomal markers, Lysotracker Red and acridine orange, demonstrated that lysosomal Pgp potentiated Dp44mT-mediated lysosomal damage since the Pgp inhibitors prevented this effect only in Pgp-expressing cells (FIG. 7A-F). The present inventors studies also demonstrated that Cu[Dp44mT] acted to induce lysosomal permeabilization and led to apoptosis. However, Pgp was not implicated in this mechanism. In the current investigation, Cu[Dp44mT] rapidly induced a decrease in lysosomal integrity (after 30 min) in Pgp-expressing cells relative to their non-Pgp expressing counterparts which could be reversed by a Pgp inhibitor (FIG. 7A-E).

Similarly, the Dp44mT ligand alone also compromised lysosomal integrity but only after a 24 h incubation (FIG. 7F). The explanation for the delayed activity of Dp44mT could be due to: (1) that in accordance with Pgp-ATPase activity measurements, Cu[Dp44mT], was a better substrate than Dp44mT alone (FIG. 4A), allowing more rapid accumulation in the lysosome and subsequent damage; and (2) in contrast to the pre-formed Cu[Dp44mT] complex, Dp44mT would need to acquire free copper from copper containing proteins being metabolized in the lysosome before redox activity can be initiated as the cytotoxic copper complex (FIG. 3). It is well known that the lysosome plays a role in autophagy¹⁷ and the recycling of metal ions including copper and that the permeabilization of this organelle plays a crucial role in the apoptosis induced by Dp44mT. Hence, ROS-generating agents such as Dp44mT can hijack lysosomal Pgp to selectively overcome drug resistance by inducing more lysosomal damage in Pgp-expressing cells (FIG. 1).

For the first time, the studies discussed herein demonstrate a key role for lysosomal Pgp in overcoming drug resistance and offer a mechanistic explanation for the potentiated cytotoxicity reported in cells possessing MDR.

Furthermore, as part of the increased metal metabolism observed in neoplastic cells, lysosomes in tumor cells contain greater quantities of metals, providing, in part, an explanation for the selectivity of these agents against cancer cells relative to their normal counterparts. Hence, the studies demonstrate that hijacking Pgp in the lysosomal membrane of cells expressing this protein can increase uptake of agents that induce lysosomal oxidative stress (FIG. 7).

Finally, it is notable that the sensitizing action of Pgp on Dp44mT-mediated cytotoxicity was dependent on three characteristics of this agent, namely (1) it must be a P-gp substrate; (2) it must be able to become charged at acidic pH due to its ionization properties leading to its accumulation in the lysosome; and (3) the agent must cause marked redox stress in the acidic lysosomal environment leading to cytotoxic ROS that induces lysosomal membrane permeabilization and subsequently apopotosis and cell death. Considering these 3 points, it is notable that DOX possesses (1) and (2), but unlike Dp44mT, DOX does not form copper complexes that possess marked redox activity which lead to lysosomal permeabilization. This study offers a novel strategy that can be utilized in the design of new agents such as Dp44mT and its analogues that can selectively overcome drug resistance.

It will be understood that the invention disclosed and defined in this specification extends to all alternative combinations of two or more of the individual features mentioned or evident from the text or drawings. All of these different combinations constitute various alternative aspects of the invention.

### REFERENCES

1. Bacchi, A., Came M., Costa, M., Pelagatti, P., Pelizzi, C. & Pelizzi, G. Verstile ligand behaviour of phenyl 2-pyridyl ketone benzoylhydrazone in palladium(II) complexes. J Chem Soc, Dalton Trans 4239-4244 (1996).
2. Bacchi, A., Bonini, A., Carcelli, M., Ferraro, F., Leporati, E., Pelizzi, C. & Pelizzi, G. Chelating behaviour of methyl 2-pyridyl ketone carbono- and thiocarbonohydrazones in copper(II) and zinc(II) complexes. J Chem Soc, Dalton Trans 2699-2704 (1996).
3. Johnson, D. K., Murphy, T. B., Rose, N. J., Goodwin, W. H. & Pickart, L. Cytotoxic chelators and chelates 1. Inhibition of DNA synthesis in cultured rodent and human cells by aroylhydrazones and by a copper(II) complex of salicylaldehyde benzoyl hydrazone. Inorg Chim Act 67, 159-165 (1982).
4. Berge, S. M., Bighiey, L. D. & Monkhouse, D. C. Pharmaceutical salts. J Pharm Sci 66, 1-19 (1977).
5. Whitnall, M., Howard, J., Ponka, P. & Richardson, D. R. A class of iron chelators with a wide spectrum of potent antitumor activity that overcomes resistance to chemotherapeutics. Proc Natl Acad Sci USA 103, 14901-14906 (2006).
6. Ludwig, J. A., et al. Selective toxicity of NSC73306 in MDR1-positive cells as a new strategy to circumvent multidrug resistance in cancer. Cancer Res 66, 4808-4815 (2006).
7. Shen, D. W., at al. Multiple drug-resistant human KB carcinoma cells independently selected for highlevel resistance to colchicine, adriamycin, or vinblastine show changes in expression of specific proteins. J Biol Chem 261, 7762-7770 (1986).
8. Holland, M. L., Allen, J. D. & Arnold, J. C. Interaction of plant cannabinoids with the multidrug transporter ABCC1 (MRP1). Eur J Pharmacol 591, 128-131 (2008).
9. Kalinowski, D. S., et al. Design, synthesis, and characterization of novel iron chelators: structure-activity relationships of the 2-benzoylpyridine thiosemicarbazone series and their 3-nitrobenzoyl analogues as potent antitumor agents. J Med Chem 50, 3716-3729 (2007).
10. Suarez, Y., et al, Kahalalide F, a new marine-derived compound, induces oncosis in human prostate and breast cancer cells. Mol Cancer Ther 2, 863-872 (2003).
11. Jansson, P. J., Hawkins, C. L., Lovejoy, D. B. & Richardson, D. R. The iron complex of Dp44mT is redoxactive and induces hydroxyl radical formation: an EPR study. J Inorg Biochem 104, 1224-1228 (2010).
12. Turk, D., et al. Identification of compounds selectively killing multidrug-resistant cancer cells. Cancer Res 69, 8293-8301 (2009).
13. Kappus, H. & Sies, H. Toxic drug effects associated with oxygen metabolism: redox cycling and lipid peroxidation. Experientia 37, 1233-1241 (1981).
14. Pluchino, K. M., Hall, M. D., Goldsborough, A. S., Callaghan, R. & Gottesman, M. M. Collateral sensitivity as a strategy against cancer multidrug resistance. Drug Resist Updat 15, 98-105 (2012).
15. Lovejoy, D. B., et al. Antitumor activity of metal-chelating compound Dp44mT is mediated by formation of a redox-active copper complex that accumulates in lysosomes. Cancer Res 71, 5871-5880 (2011).
16. Antoine, J. C., Goud, B., Jouanne, C., Maurice, M. & Feldmann, G. Ammonium chloride, methylamine and chloroquine reversibly inhibit antibody secretion by plasma cells. Biol Cell 55, 41-54 (1985).
17. Kurz, T., Terman, A. & Brunk, U. T. Autophagy, ageing and apoptosis: the role of oxidative stress and lysosomal iron. Arch Biochem Biophys 462, 220-230 (2007).
18. Richardson, D. R., Sharpe, P. C., Lovejoy, D. B., et al. Dipyridyl thiosemicarbazone chelators with potent and selective antitumor activity form iron complexes with redox activity. J Med Chem 49, 6510-21 (2006).
19. Bernhardt, P. V., Sharpe, P. C., Islam, M., Lovejoy, D. B., Kalinowski, D. S., Richardson, D. R. Iron chelators of the dipyridylketone thiosemicarbazone class: precomplexation and transmetalation effects on anticancer activity. J Med Chem 52, 407-15 (2009).
20. Jansson, P. J., Sharpe, P. C., Bernhardt, P. V., Richardson, D. R. Novel thiosemicarbazones of the ApT and DpT series and their copper complexes: identification of pronounced redox activity and characterization of their antitumor activity. J Med Chem 53, 5759-69 (2010).
21. Yamagishi, T., Sahni, S., Sharp, D. M., Arvind, A., Jansson, P. J., Richardson, D. R. P-Glycoprotein Mediates Drug Resistance via a Novel Mechanism Involving Lysosomal Sequestration. J Biol Chem 288, 31761-71 (2013).

1. A method of treating a cancer in an individual comprising:
administering an effective amount of a compound to an individual who has cancer, the cancer including a cancer cell that includes an active efflux mechanism for efflux of an anti-cancer agent from a cancer cell;
wherein the compound is a substrate of the active efflux mechanism and the compound is able to form a chelation complex with a metal species in the cancer cell, the chelation complex being cytotoxic to the cancer cell,
thereby treating the cancer in the individual.

2. The method of claim 1, wherein the compound forms the chelation complex with the metal species in the lysosome of the cancer cell.

3. The method of claim 1, wherein the active efflux mechanism is mediated by a polypeptide that is a member of the ABC gene superfamily.

4. The method of claim 3, wherein the mechanism is mediated by P-glycoprotein (ABCB1).

5. The method of claim 4, wherein the cancer cell expresses Pgp on one or more of the cell surface, endosome and lysosome.

6. The method of claim 1 further comprising an initial step of:
selecting an individual for treatment of cancer, wherein the individual has received chemotherapy or radiotherapy for the cancer.

7. The method of claim 6, wherein the cancer is resistant to a chemotherapeutic drug previously administered.

8. The method of claim 7, wherein the cancer is resistant to multiple chemotherapeutic drugs.

9. The method of claim 1, wherein the cancer cell has acquired multiple drug resistance, and wherein the cancer cell expresses Pgp on the cell surface.

10. A method according to claim 1 including the step of selecting an individual for treatment of cancer, wherein the individual is selected on the basis of the presence of Pgp transporters on the cell surface of a cancer cell of the individual.

11. The method of claim 1, wherein the metal ion species is a copper or iron ion species.

12. The method of claim 1, wherein the compound is a compound of Formula 1: 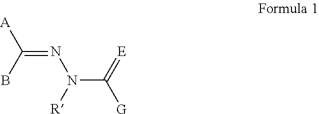
wherein:
A is a monocyclic or polycyclic substituted or unsubstituted 5 or 6-membered heteroaryl group;
B is selected from the group consisting of: a substituted or unsubstituted alkyl group, a substituted or unsubstituted alkene group, a substituted or unsubstituted alkyne group, a monocyclic or polycyclic substituted or unsubstituted cycloalkyl group, a monocyclic or polycyclic substituted or unsubstituted heterocycloalkyl group, a monocyclic or polycyclic substituted or unsubstituted aryl group, or a monocyclic or polycyclic substituted or unsubstituted heteroaryl group;
R1 is any group that is exchangeable upon binding of the compound to a metal ion;
E is O or S;
G is selected from the group consisting of: a substituted or unsubstituted amine group, a substituted or unsubstituted alkyl group, a substituted or unsubstituted alkene group, a substituted or unsubstituted alkyne group, a monocyclic or polycyclic substituted or unsubstituted cycloalkyl group, a monocyclic or polycyclic substituted or unsubstituted heterocycloalkyl group, a monocyclic or polycyclic substituted or unsubstituted aryl group, or a monocyclic or polycyclic substituted or unsubstituted heteroaryl group.

13. The method of claim 12, wherein A is represented by Formula 2: 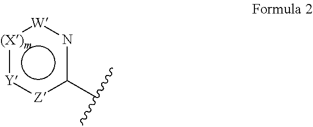
wherein W′, X′, Y′, and Z′ are independently selected from the group consisting of: N, CH, S and O; and
wherein the total number of heteroatoms is 1, 2, or 3; and m is 0 or 1.

14. The method of claim 5, wherein B is represented by Formula 3: 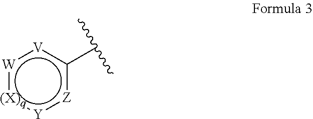
wherein V, W, X, Y, and Z are independently selected from the group consisting of: N, CH, S and O; and
wherein the total number of heteroatoms is 1, 2, or 3; and q is 0 or 1.

15. The method of claim 12, wherein A is a substituted or unsubstituted pyridine.

16. The method of claim 12, wherein B is a substituted or unsubstituted pyridine.

17. The method of claim 12, wherein G is selected from the group consisting of: NH₂, NHR′, or NR′R″, wherein R′ and R″ are independently selected from the group consisting of: a substituted or unsubstituted amine group, a substituted or unsubstituted alkyl group, a monocyclic or polycyclic substituted or unsubstituted heterocycloalkyl group, a monocyclic or polycyclic substituted or unsubstituted aryl group, a monocyclic or polycyclic substituted or unsubstituted cycloalkyl group, a monocyclic or polycyclic substituted or unsubstituted heteroaryl group.

18. The method of claim 1, wherein the compound is a DpT derivative selected from the group consisting of: di-2-pyridylketone 4,4-diphenylcarboxaldehyde semicarbazone (PK44 pH), di-2-pyridylketone 4-methyl-3-thiosemicarbazone (Op4mT), di-2-pyridylketone 4,4-dimethyl-3-thiosemicarbazone (Dp44mT), di-2-pyridylketone 4-ethyl-3-thiosemicarbazone (Dp4eT), di-2-pyridylketone 4-allyl-3-thiosemicarbazone (Op4aT), di-2-pyridylketone 4-phenyl-3-thiosemicarbazone (Dp4pT), and di-2-pyridylketone 4,4-diphenylcarboxaldehyde thiosemicarbazone (PK44pTH).

19. The method of claim 1, wherein the compound is a PKIH derivative selected from the group consisting of: di-2-pyridylketone isonicotinoyl hydrazone (PKIH); di-2-pyridylketone benzoyl hydrazone (PKBH), di-2-pyridylketone 4-hydroxybenzoyl hydrazone (PKHH), di-2-pyridylketone 3-bromobenzoyl hydrazine (PBBH), di-2-pyridylketone 4-aminobenzoyl hydrazone (PKAH), di-2-pyridylketone 2-thiophenecarboxaldehyde hydrazone (PKTH), di-2-pyridylketone octanoic hydrazine (PKoctH), di-2 pyridylketone isonicotinoyl thiohydrazone (PKITH), di-2-pyridylketone benzoyl thiohydrazone (PKBTH), di-2-pyridylketone 4-hydroxybenzoyl thiohydrazone (PKHTH), di-2-pyridylketone 3-bromobenzoyl thiohydrazone (PBBTH), di-2-pyridylketone 4-aminobenzoyl thiohydrazone (PKATH), di-2-pyridylketone 2-thiophenecarboxaldehyde thiohydrazone (PKTTH), and di-2-pyridylketone octanoic thiohydrazone (PKoctTH).

20. The method of claim 11, wherein the copper ion species is selected from the group consisting of: CuCl₂, Cu(NO₃)₂, CuSO₄, Cu(OAc)₂, and Cu(ClO₄)₂.

21. The method of claim 11, wherein the iron ion species is selected from the group consisting of: FeCl₃, Fe(NO₃)₃, FeSO₄, Fe(OAc)₃, and Fe₂(ClO₄)₃.

22.-45. (canceled)
